(19) United States
(12) Patent Application Publication
(10) Pub. No.: US 2011/0263629 A1
(43) Pub. Date: Oct. 27, 2011

(54) AMIDES OF DIAZABICYCLOOCTANES AND USES THEREOF

(75) Inventors: Jon-Paul Strachan, Burlington, NC (US); Balwinder Singh Bhatti, Winston-Salem, NC (US); Anatoly Mazurov, Greensboro, NC (US); Jozef Klucik, Marietta, GA (US); Yunde Xiao, Clemmons, NC (US); Philip S. Hammond, Pinnacle, NC (US); David Kombo, Winston-Salem, NC (US); Lan Miao, Advance, NC (US); Jason Daniel Speake, Winston-Salem, NC (US); Daniel Yohannes, Winston-Salem, NC (US)

(73) Assignee: Targacept, Inc., Winston-Salem, NC (US)

(21) Appl. No.: 13/061,972

(22) PCT Filed: Sep. 2, 2009
(86) PCT No.: PCT/US2009/055718

§ 371 Date: Jul. 6, 2011

Related U.S. Application Data
(60) Provisional application No. 61/094,647, filed on Sep. 5, 2008.

(51) Int. Cl. A61K 31/439; A61P 25/00; A61P 25/28; A61P 25/18; A61P 3/04; A61P 3/00; A61P 37/06; A61P 25/30; A61P 25/04; C07D 471/08; A61P 29/00

(52) U.S. Cl. 514/278; 546/113; 546/15; 514/300

(57) ABSTRACT

The present invention relates to compounds that bind to and modulate the activity of neuronal nicotinic acetylcholine receptors, to processes for preparing these compounds, to pharmaceutical compositions containing these compounds, and to methods of using these compounds for treating a wide variety of conditions and disorders, including those associated with dysfunction of the central nervous system (CNS).

### FIELD OF THE INVENTION

The present invention relates to compounds that bind to and modulate the activity of neuronal nicotinic acetylcholine receptors, to processes for preparing these compounds, to pharmaceutical compositions containing these compounds, and to methods of using these compounds for treating a wide variety of conditions and disorders, including those associated with dysfunction of the central nervous system (CNS).

### BACKGROUND OF THE INVENTION

The therapeutic potential of compounds that target neuronal nicotinic receptors (NNRs), also known as nicotinic acetylcholine receptors (nAChRs), has been the subject of several reviews. See, for example, Breining et al., Ann. Rep. Med. Chem. 40: 3 (2005), Hogg and Bertrand, Curr. Drug Targets: CNS Neurol. Disord. 3: 123 (2004), Suto and Zacharias, Expert Opin. Ther. Targets 8: 61 (2004), Dani et al., Bioorg. Med. Chem. Lett. 14: 1837 (2004), Bencherif and Schmitt, Curr. Drug Targets: CNS Neurol. Disord. 1: 349 (2002), each incorporated by reference with regard to such teaching. Among the kinds of indications for which NNR ligands have been proposed as therapies are cognitive disorders, including Alzheimer's disease, attention deficit disorder, and schizophrenia (Newhouse et al., Curr. Opin. Pharmacol. 4: 36 (2004), Levin and Rezvani, Curr. Drug Targets: CNS Neurol. Disord. 1: 423 (2002), Graham et al., Curr. Drug Targets: CNS Neurol. Disord. 1: 387 (2002), Ripoll et al., Curr. Med. Res. Opin. 20(7): 1057 (2004), and McEvoy and Allen, Curr. Drug Targets: CNS Neurol. Disord. 1: 433 (2002)); pain and inflammation (Decker et al., Curr. Top. Med. Chem. 4(3): 369 (2004), Vincler, Expert Opin. Invest. Drugs 14(10): 1191 (2005), Jain, Curr. Opin. Inv. Drugs 5: 76 (2004), Miao et al., Neuroscience 123: 777 (2004)); depression and anxiety (Shytle et al., Mol. Psychiatry 7: 525 (2002), Damaj et al., Mol. Pharmacol. 66: 675 (2004), Shytle et al., Depress. Anxiety 16: 89 (2002)); neurodegeneration (O'Neill et al., Curr. Drug Targets: CNS Neurol. Disord. 1: 399 (2002), Takata et al., J. Pharmacol. Exp. Ther. 306: 772 (2003), Marrero et al., J. Pharmacol. Exp. Ther. 309: 16 (2004)); Parkinson's disease (Jonnala and Buccafusco, J. Neurosci. Res. 66: 565 (2001)); addiction (Dwoskin and Crooks, Biochem. Pharmacol. 63: 89 (2002), Coe et al., Bioorg. Med. Chem. Lett. 15(22): 4889 (2005)); obesity (Li et al., Curr. Top. Med. Chem. 3: 899 (2003)); and Tourette's syndrome (Sacco et al., J. Psychopharmacol. 18(4): 457 (2004), Young et al., Clin. Ther. 23(4): 532 (2001)), each of these references incorporated by reference with regard to the nexus of the receptor and the named indication(s).

A limitation of some nicotinic compounds is that they are associated with various undesirable side effects due to non-specific binding to multiple nAChR subtypes. For example, binding to and stimulation of muscle and ganglionic nAChR subtypes can lead to side effects which can limit the utility of a particular nicotinic binding compound as a therapeutic agent. The compounds of the present invention exhibit a high degree of binding to the ′4β2 nAChR subtype and lower affinity for α7, ganglionic and muscle nAChR subtypes. Thus, these compounds can serve as therapeutic modulators of α4β2 nAChRs in patients in need of such treatment, without producing side effects caused by non-specific nAChR subtype binding.

### SUMMARY OF THE INVENTION

The present invention includes compounds of either Formula I or Formula II:

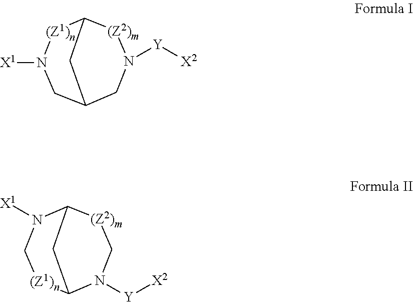

wherein:
Y is C(O), C(S), or S(O)_{q};
q is 1 or 2;
Z¹ is methylene and n is 0 or 1;
Z² is methylene and m is 0 or 1;
when n is 0, then m is 1;
when m is 0, then n is 1;
X¹ is hydrogen or C₁₋₆ alkyl;
X² is R^{I}, OR^{II}, or NR^{III}R^{IV};
when Y is C(O), then R^{I} is hydrogen, optionally substituted C₁₋₆ alkyl, optionally substituted C₃₋₈ cycloalkyl, optionally substituted C₂₋₆ alkenyl, optionally substituted C₃₋₈ cycloalkenyl, optionally substituted C₂₋₆ alkynyl, optionally substituted heterocyclyl, optionally substituted aryl, optionally substituted arylalkyl, or optionally substituted heteroarylalkyl;
when Y is C(S) or S(O)_{q}, then R^{I} is hydrogen, optionally substituted C₁₋₆ alkyl, optionally substituted C₃₋₈ cycloalkyl, optionally substituted C₂₋₆ alkenyl, optionally substituted C₃₋₈ cycloalkenyl, optionally substituted C₂₋₆alkynyl, optionally substituted heterocyclyl, optionally substituted aryl, optionally substituted heteroaryl, optionally substituted arylalkyl, or optionally substituted heteroarylalkyl;
R^{II} is hydrogen, optionally substituted C₁₋₆ alkyl, optionally substituted C₃₋₈ cycloalkyl, optionally substituted C₂₋₆ alkenyl, optionally substituted C₃₋₈ cycloalkenyl, optionally substituted C₂₋₆ alkynyl, optionally substituted heterocyclyl, optionally substituted aryl, optionally substituted heteroaryl, optionally substituted arylalkyl, or optionally substituted heteroarylalkyl; and
each of R^{III and R}^{IV} are individually hydrogen, optionally substituted C₁₋₆ alkyl, optionally substituted C₃₋₈ cycloalkyl, optionally substituted C₂₋₆ alkenyl, optionally substituted C₃₋₈cycloalkenyl, optionally substituted C₂₋₆ alkynyl, optionally substituted heterocyclyl, optionally substituted aryl, optionally substituted heteroaryl, optionally substituted arylalkyl, or optionally substituted heteroarylalkyl;
or R^{III} and R^{IV} can combine with the nitrogen to which they are attached to form a 3- to 8-membered ring that may contain one or more degrees of unsaturation and may contain one or more additional heteroatom selected from N, O, or S;
where the term “optionally substituted” refers to optional substitution of one or more hydrogen atoms by a substituent independently selected from C₁₋₆ alkyl, C₃₋₈ cycloalkyl, heterocyclyl, aryl, heteroaryl, halogen, OR^{V}, NR^{V}R^{VI}, C₁₋₆ haloalkyl, —CN, —NO₂, —C₂R^{V}, —SR^{V}, —N₃, —C(═O)NR^{V}R^{VI}, —NR^{V}C(═O)R^{VI}, —OC(═O)NR^{V}R^{VI}, —NR^{V}C(═O)OR^{VI}, —SO₂R^{V}, —SO₂NR^{V}R^{VI}, and —NR^{V}SO₂R^{VI}, where R^{V} and R^{VI} are individually hydrogen, C₁₋₆ alkyl, C₃₋₈ cycloalkyl, heterocyclyl, aryl, or arylalkyl;
or a pharmaceutically acceptable salt thereof.

The compounds of the present invention bind with high affinity to NNRs of the α4β2 subtype and exhibit selectivity for this subtype over the α7 NNR subtype, as well as ganglion and muscle subtypes. The present invention also relates to pharmaceutically acceptable salts prepared from these compounds.

The present invention includes pharmaceutical compositions comprising a compound of the present invention or a pharmaceutically acceptable salt thereof. The pharmaceutical compositions of the present invention can be used for treating or preventing a wide variety of conditions or disorders, including those disorders characterized by dysfunction of nicotinic cholinergic neurotransmission or the degeneration of the nicotinic cholinergic neurons.

The present invention includes a method for treating or preventing disorders and dysfunctions, such as CNS disorders and dysfunctions, inflammation, inflammatory response associated with bacterial and/or viral infection, pain, metabolic syndrome, autoimmune disorders, or other disorders described in further detail herein. The present invention includes a method for modulating neovascularization. The methods involve administering to a subject a therapeutically effective amount of a compound of the present invention, including a salt thereof, or a pharmaceutical composition that includes such compounds. Additionally, the present invention includes compounds that have utility as diagnostic agents and in receptor binding studies as described herein.

The foregoing and other aspects of the present invention are explained in further detail in the detailed description and examples set forth below.

### DETAILED DESCRIPTION

### I. Compounds

The following definitions are meant to clarify, but not limit, the terms defined. If a particular term used herein is not specifically defined, such term should not be considered indefinite. Rather, terms are used within their accepted meanings.

As used throughout this specification, the preferred number of atoms, such as carbon atoms, will be represented by, for example, the phrase “Cₓ-C_{y} alkyl,” which refers to an alkyl group, as herein defined, containing the specified number of carbon atoms. Similar terminology will apply for other preferred terms and ranges as well. One embodiment of the present invention includes so-called ‘lower’ alkyl chains of one to eight, preferably one to six carbon atoms. Thus, for example, C₁-C₆ alkyl represents a lower alkyl chain as hereinabove described.

As used herein the term “alkyl” refers to a straight or branched chain hydrocarbon having one to eight carbon atoms, preferably one to six carbon atoms, which may be optionally substituted as herein further described, with multiple degrees of substitution being allowed. Examples of “alkyl” as used herein include, but are not limited to, methyl, ethyl, propyl, isopropyl, isobutyl, n-butyl, tert-butyl, isopentyl, and n-pentyl.

As used herein the term “alkenyl” refers to a straight or branched chain aliphatic hydrocarbon having two to twelve carbon atoms, preferably two to eight carbon atoms, and containing one or more carbon-to-carbon double bonds, which may be optionally substituted as herein further described, with multiple degrees of substitution being allowed. Examples of “alkenyl” as used herein include, but are not limited to, vinyl, and allyl.

As used herein the term “alkynyl” refers to a straight or branched chain aliphatic hydrocarbon having two to twelve carbon atoms, preferably two to eight carbon atoms, and containing one or more carbon-to-carbon triple bonds, which may be optionally substituted as herein further described, with multiple degrees of substitution being allowed. An example of “alkynyl” as used herein includes, but is not limited to, ethynyl.

As used herein, the term “cycloalkyl” refers to a fully saturated optionally substituted three- to twelve-membered, preferably three- to eight-membered, monocyclic, bicyclic, Spiro, or bridged hydrocarbon ring, with multiple degrees of substitution being allowed. Exemplary “cycloalkyl” groups as used herein include, but are not limited to, cyclopropyl, cyclobutyl, cyclopentyl, cyclohexyl, and cycloheptyl.

Similarly, as used herein, the terms “cycloalkenyl” and “cycloalkynyl” refer to optionally substituted, partially saturated but non-aromatic, three-to-twelve membered, preferably either five- to eight-membered or seven- to ten-membered, monocyclic, bicyclic, or bridged hydrocarbon rings, with one or more degrees of unsaturation, and with multiple degrees of substitution being allowed.

As used herein, the term “heterocycle” or “heterocyclyl” refers to an optionally substituted mono- or polycyclic ring system, optionally containing one or more degrees of unsaturation and also containing one or more heteroatoms, which may be optionally substituted as herein further described, with multiple degrees of substitution being allowed. Exemplary heteroatoms include nitrogen, oxygen, or sulfur atoms, including N-oxides, sulfur oxides, and dioxides. Preferably, the ring is three to twelve-membered, preferably three- to eight-membered and is either fully saturated or has one or more degrees of unsaturation. Such rings may be optionally fused to one or more of another heterocyclic ring(s) or cycloalkyl ring(s). Examples of “heterocyclic” groups as used herein include, but are not limited to, tetrahydrofuran, pyran, 1,4-dioxane, 1,3-dioxane, piperidine, pyrrolidine, morpholine, tetrahydrothiopyran, and tetrahydrothiophene.

As used herein, the term “aryl” refers to a univalent benzene ring or fused benzene ring system, which may be optionally substituted as herein further described, with multiple degrees of substitution being allowed. Examples of “aryl” groups as used include, but are not limited to, phenyl, 2-naphthyl, 1-naphthyl, anthracene, and phenanthrene. Preferable aryl rings have five- to ten-members.

As used herein, a fused benzene ring system encompassed within the term “aryl” includes fused polycyclic hydrocarbons, namely where a cyclic hydrocarbon with less than maximum number of noncumulative double bonds, for example where a saturated hydrocarbon ring (cycloalkyl, such as a cyclopentyl ring) is fused with an aromatic ring (aryl, such as a benzene ring) to form, for example, groups such as indanyl and acenaphthalenyl, and also includes such groups as, for non-limiting examples, dihydronaphthalene and hexahydrocyclopenta-cyclooctene.

As used herein, the term “aralkyl” refers to an “aryl” group as herein defined attached through an alkylene linker.

As used herein, the term “heteroaryl” refers to a monocyclic five to seven membered aromatic ring, or to a fused bicyclic aromatic ring system comprising two of such aromatic rings, which may be optionally substituted as herein further described, with multiple degrees of substitution being allowed. Preferably, such rings contain five- to ten-members. These heteroaryl rings contain one or more nitrogen, sulfur, and/or oxygen atoms, where N-oxides, sulfur oxides, and dioxides are permissible heteroatom substitutions. Examples of “heteroaryl” groups as used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzoxazole, benzothiophene, indole, indazole, benzimidazole, imidazopyridine, pyrazolopyridine, and pyrazolopyrimidine.

As used herein, the term “heteroaralkyl” refers to an “heteroaryl” group as herein defined attached through an alkylene linker.

As used herein the term “halogen” refers to fluorine, chlorine, bromine, or iodine.

As used herein the term “haloalkyl” refers to an alkyl group, as defined herein, that is substituted with at least one halogen. Examples of branched or straight chained “haloalkyl” groups as used herein include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups such as —CF₃.

As used herein the term “alkoxy” refers to a group —OR^{a}, where R^{a} is alkyl as defined above.

As used herein the term “nitro” refers to a group —NO₂.

As used herein the term “cyano” refers to a group —CN.

As used herein the term “azido” refers to a group —N₃.

As used herein “amino” refers to a group —NR^{a}R^{b}, where each of R^{a} and R^{b} individually is hydrogen, alkyl, alkenyl, alkynyl, cycloalkyl, aryl, heterocylcyl, or heteroaryl. As used herein, when either R^{a} or R^{b} is other than hydrogen, such a group may be referred to as a “substituted amino” or, for example if R^{a} is H and R^{b} is alkyl, as an “alkylamino.”

As used herein, the term “hydroxyl” refers to a group —OH.

One embodiment of the present invention includes a compound as represented by either Formula I or Formula II:

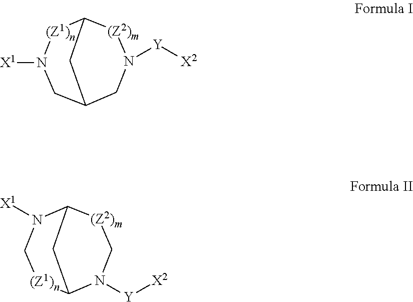

wherein:
Y is C(O), C(S), or S(O)_{q};
q is 1 or 2;
Z¹ is methylene and n is 0 or 1;
Z² is methylene and m is 0 or 1;
when n is 0, then m is 1;
when m is 0, then n is 1;
X¹ is hydrogen or C₁₋₆ alkyl;
X² is R^{I}, OR^{II}, or NR^{III}R^{IV};
when Y is C(O), then R^{I} is hydrogen, optionally substituted C₁₋₆ alkyl, optionally substituted C₃₋₈ cycloalkyl, optionally substituted C₂₋₆ alkenyl, optionally substituted C₃₋₈cycloalkenyl, optionally substituted C₂₋₆ alkynyl, optionally substituted heterocyclyl, optionally substituted aryl, optionally substituted arylalkyl, or optionally substituted heteroarylalkyl;
when Y is C(S) or S(O)_{q}, then R^{I} is hydrogen, optionally substituted C₁₋₆ alkyl, optionally substituted C₃₋₈ cycloalkyl, optionally substituted C₂₋₆ alkenyl, optionally substituted C₃₋₈ cycloalkenyl, optionally substituted C₂₋₆ alkynyl, optionally substituted heterocyclyl, optionally substituted aryl, optionally substituted heteroaryl, optionally substituted arylalkyl, or optionally substituted heteroarylalkyl;
R^{II} is hydrogen, optionally substituted C₁₋₆ alkyl, optionally substituted C₃₋₈ cycloalkyl, optionally substituted C₂₋₆ alkenyl, optionally substituted C₃₋₈ cycloalkenyl, optionally substituted C₂₋₆alkynyl, optionally substituted heterocyclyl, optionally substituted aryl, optionally substituted heteroaryl, optionally substituted arylalkyl, or optionally substituted heteroarylalkyl; and
each of R^{III} and R^{IV} are individually hydrogen, optionally substituted C₁₋₆ alkyl, optionally substituted C₃₋₈ cycloalkyl, optionally substituted C₂₋₆ alkenyl, optionally substituted C₃₋₈ cycloalkenyl, optionally substituted C₂₋₆ alkynyl, optionally substituted heterocyclyl, optionally substituted aryl, optionally substituted heteroaryl, optionally substituted arylalkyl, or optionally substituted heteroarylalkyl;
or R^{III} and R^{IV} can combine with the nitrogen to which they are attached to form a 3- to 8-membered ring that may contain one or more degrees of unsaturation and may contain one or more additional heteroatom selected from N, O, or S;
where the term “optionally substituted” refers to optional substitution of one or more hydrogen atoms by a substituent independently selected from C₁₋₆ alkyl, C₃₋₈ cycloalkyl, heterocyclyl, aryl, heteroaryl, halogen, OR^{V}, NR^{V}R^{VI}, C₁₋₆ haloalkyl, —CN, —NO₂, —C₂R^{V}, —SR^{V}, —N₃, —C(═O)NR^{V}R^{VI}, —NR^{V}C(═O)R^{VI}, —OC(═O)NR^{V}R^{VI}, —NR^{V}C(═O)OR^{VI}, —SO₂R^{V}, —SO₂NR^{V}R^{VI}, and —NR^{V}SO₂R^{VI}, where R^{V} and R^{VI} are individually hydrogen, C₁₋₆ alkyl, C₃₋₈ cycloalkyl, heterocyclyl, aryl, or arylalkyl;
or a pharmaceutically acceptable salt thereof.

One embodiment of the present invention includes a compound selected from the group consisting of:
3-(cyclopentene-4-ylcarbonyl)-3,6-diazabicyclo[3.2.1]octane,
3-(cyclopropylcarbonyl)-3,6-diazabicyclo[3.2.1]octane,
3-(trifluoroacetyl)-3,6-diazabicyclo[3.2.1]octane,
3-(methoxycarbonyl)-3,6-diazabicyclo[3.2.1]octane,
(1S,5S)-3-(cyclopropylcarbonyl)-3,6-diazabicyclo[3.2.1]octane,
(1S,5S)-3-(cyclopentene-4-ylcarbonyl)-3,6-diazabicyclo[3.2.1]octane,
(1S,5S)-3-(tetrahydrofuran-3-ylcarbonyl)-3,6-diazabicyclo[3.2.1]octane,
(1S,5S)-3-(trifluoroacetyl)-3,6-diazabicyclo[3.2.1]octane,
(1R,5R)-3-(cyclopropylcarbonyl)-3,6-diazabicyclo[3.2.1]octane,
(1R,5R)-3-(cyclopentene-4-ylcarbonyl)-3,6-diazabicyclo[3.2.1]octane,
(1S,5S)-3-(cyclobutylcarbonyl)-3,6-diazabicyclo[3.2.1]octane,
(1S,5S)-3-(acetyl)-3,6-diazabicyclo[3.2.1]octane,
(1R,5R)-3-(trifluoroacetyl)-3,6-diazabicyclo[3.2.1]octane,
(1R,5R)-3-(acetyl)-3,6-diazabicyclo[3.2.1]octane,
(1R,5R)-3-(cyclobutylcarbonyl)-3,6-diazabicyclo[3.2.1]octane,
(1S,5S)-3-(2,2-dimethylpropanoyl)-3,6-diazabicyclo[3.2.1]octane,
(1R,5R)-3-(2,2-dimethylpropanoyl)-3,6-diazabicyclo[3.2.1]octane,
(1S,5S)-3-(methoxycarbonyl)-3,6-diazabicyclo[3.2.1]octane,
(1S,5S)-3-(isopropoxycarbonyl)-3,6-diazabicyclo[3.2.1]octane,
(1S,5S)-3-(4-fluorophenoxycarbonyl)-3,6-diazabicyclo[3.2.1]octane,
(1S,5S)-3-(2-methoxyethoxycarbonyl)-3,6-diazabicyclo[3.2.1]octane,
(1R,5R)-3-(2-methoxyethoxycarbonyl)-3,6-diazabicyclo[3.2.1]octane,
(1R,5R)-3-(4-fluorophenoxycarbonyl)-3,6-diazabicyclo[3.2.1]octane,
(1S,5S)-3-(propanoyl)-3,6-diazabicyclo[3.2.1]octane,
(1S,5S)-3-(butanoyl)-3,6-diazabicyclo[3.2.1]octane,
(1S,5S)-3-(2-methylpropanoyl)-3,6-diazabicyclo[3.2.1]octane,
(1S,5S)-3-(cyclopentylcarbonyl)-3,6-diazabicyclo[3.2.1]octane,
E-(1S,5S)-3-(3-pentenoyl)-3,6-diazabicyclo[3.2.1]octane,
(1S,5S)-3-(ethoxycarbonyl)-3,6-diazabicyclo[3.2.1]octane,
(1S,5S)-3-(methylsulfonyl)-3,6-diazabicyclo[3.2.1]octane,
(1S,5S)-3-(isopropylsulfonyl)-3,6-diazabicyclo[3.2.1]octane,
(1S,5S)-3-(phenylsulfonyl)-3,6-diazabicyclo[3.2.1]octane,
(1S,5S)-3-(4-fluorophenylsulfonyl)-3,6-diazabicyclo[3.2.1]octane,
(1S,5S)-3-(4-chlorophenylsulfonyl)-3,6-diazabicyclo[3.2.1]octane,
(1S,5S)-3-(4-methoxyphenylsulfonyl)-3,6-diazabicyclo[3.2.1]octane,
(1S,5S)-3-(2-methylcyclopropylcarbonyl)-3,6-diazabicyclo[3.2.1]octane,
(1S,5S)-3-(2,2-dimethylcyclopropylcarbonyl)-3,6-diazabicyclo[3.2.1]octane,
(1S,5S)-3-(2,2-difluorocyclopropylcarbonyl)-3,6-diazabicyclo[3.2.1]octane,
(1S,5S)-3-(spiro[2.3]hexan-1-ylcarbonyl)-3,6-diazabicyclo[3.2.1]octane,
(1S,5S)-3-(pentanoyl)-3,6-diazabicyclo[3.2.1]octane,
(1R,5R)-3-(isopropoxycarbonyl)-3,6-diazabicyclo[3.2.1]octane,
(1R,5R)-3-(methoxycarbonyl)-3,6-diazabicyclo[3.2.1]octane,
(1R,5R)-3-(ethoxycarbonyl)-3,6-diazabicyclo[3.2.1]octane,
(1S,5S)-3-(N-ethylcarbamoyl)-3,6-diazabicyclo[3.2.1]octane,
(1S,5S)-3-(N-allylcarbamoyl)-3,6-diazabicyclo[3.2.1]octane,
(1S,5S)-3-(N-(4-fluorphenyl)carbamoyl)-3,6-diazabicyclo[3.2.1]octane,
(1S,5S)-3-(N-allylthiocarbamoyl)-3,6-diazabicyclo[3.2.1]octane,
(1S,5S)-3-(3-methylbutanoyl)-3,6-diazabicyclo[3.2.1]octane,
(1S,5S)-3-(n-propoxycarbonyl)-3,6-diazabicyclo[3.2.1]octane,
(1R,5R)-3-(cyclopropylcarbonyl)-6-methyl-3,6-diazabicyclo[3.2.1]octane,
(1S,5S)-3-(2,2,3,3-tetramethylcyclopropylcarbonyl)-3,6-diazabicyclo[3.2.1]octane,
(1S,5S)-3-(cis-2-fluorocyclopropylcarbonyl)-3,6-diazabicyclo[3.2.1]octane,
(1S,5S)-3-(trans-2-fluorocyclopropylcarbonyl)-3,6-diazabicyclo[3.2.1]octane,
(1S,5S)-3-(3-fluoropropanoyl)-3,6-diazabicyclo[3.2.1]octane,
(1S,5S)-3-(3,3,3-trifluoropropanoyl)-3,6-diazabicyclo[3.2.1]octane,
(1S,5S)-3-(ethylsulfonyl)-3,6-diazabicyclo[3.2.1]octane,
(1S,5S)-3-(n-propylsulfonyl)-3,6-diazabicyclo[3.2.1]octane,
(1S,5S)-3-(n-butylsulfonyl)-3,6-diazabicyclo[3.2.1]octane,
6-(cyclopropylcarbonyl)-3,6-diazabicyclo[3.2.1]octane,
6-(cyclopentene-4-ylcarbonyl)-3,6-diazabicyclo[3.2.1]octane,
(1S,5R)-6-(cyclopropylcarbonyl)-3,6-diazabicyclo[3.2.1]octane,
(1S,5R)-6-(cis-2-fluorocyclopropylcarbonyl)-3,6-diazabicyclo[3.2.1]octane,
(1S,5R)-6-(trans-2-fluorocyclopropylcarbonyl)-3,6-diazabicyclo[3.2.1]octane,
(1S,5R)-6-(cyclobutylcarbonyl)-3,6-diazabicyclo[3.2.1]octane,
(1S,5R)-6-(butanoyl)-3,6-diazabicyclo[3.2.1]octane,
(1S,5R)-6-(2-methylproanoyl)-3,6-diazabicyclo[3.2.1]octane,
(1S,5R)-6-(propanoyl)-3,6-diazabicyclo[3.2.1]octane,
(1S,5R)-6-(methoxycarbonyl)-3,6-diazabicyclo[3.2.1]octane,
(1S,5R)-6-(ethoxycarbonyl)-3,6-diazabicyclo[3.2.1]octane,
(1S,5R)-6-(acetyl)-3,6-diazabicyclo[3.2.1]octane,
(1R,5S)-6-(cyclopropylcarbonyl)-3,6-diazabicyclo[3.2.1]octane,
(1R,5S)-6-(cis-2-fluorocyclopropylcarbonyl)-3,6-diazabicyclo[3.2.1]octane,
(1R,5S)-6-(trans-2-fluorocyclopropylcarbonyl)-3,6-diazabicyclo[3.2.1]octane,
(1R,5S)-6-(cyclobutylcarbonyl)-3,6-diazabicyclo[3.2.1]octane,
(1R,5S)-6-(2-methylpropanoyl)-3,6-diazabicyclo[3.2.1]octane,
(1R,5S)-6-(3-fluoropropanoyl)-3,6-diazabicyclo[3.2.1]octane,
(1R,5S)-6-(methoxycarbonyl)-3,6-diazabicyclo[3.2.1]octane,
(1R,5S)-6-(ethoxycarbonyl)-3,6-diazabicyclo[3.2.1]octane, and
(1R,5S)-6-(acetyl)-3,6-diazabicyclo[3.2.1]octane,
or a pharmaceutically acceptable salt thereof

One embodiment of the present invention includes use of a compound of the present invention in the manufacture of a medicament.

One embodiment of the present invention includes a method for the treatment or prevention of a variety of disorders and dysfunctions, comprising administering to a mammal in need of such treatment, a therapeutically effective amount of the compound of the present invention. More specifically, the disorder or dysfunction may be selected from the group consisting of CNS disorders, inflammation, inflammatory response associated with bacterial and/or viral infection, pain, metabolic syndrome, autoimmune disorders or other disorders described in further detail herein. One embodiment of the present invention includes a method for modulating neovascularization. Another embodiment of the present invention includes compounds that have utility as diagnostic agents and in receptor binding studies as described herein.

Additionally, these compounds may also have utility as diagnostic agents and in receptor binding studies as described herein.

One embodiment of the present invention includes a pharmaceutical composition comprising a therapeutically effective amount of a compound of the present invention and one or more pharmaceutically acceptable carrier.

One embodiment of the present invention includes the use of a compound of the present invention in the manufacture of a medicament for treatment of central nervous system disorders and dysfunctions.

Another embodiment of the present invention includes a compound as herein described with reference to any one of the Examples.

Another embodiment of the present invention includes a compound of the present invention for use as an active therapeutic substance.

Another embodiment of the present invention includes a compound of the present invention for use to modulate an NNR in a subject in need thereof.

Another embodiment of the present invention includes a compound of the present invention for use in the treatment or prevention of conditions or disorders mediated by NNR.

Another embodiment of the present invention includes a use of a compound of the present invention in the manufacture of a medicament for use of modulating NNR in a subject in need thereof.

Another embodiment of the present invention includes a use of a compound of the present invention in the manufacture of a medicament for use in the treatment or prevention of conditions or disorders mediated by NNR.

Another embodiment of the present invention includes a method of modulating NNR in a subject in need thereof through the administration of a compound of the present invention.

The scope of the present invention includes combinations of embodiments.

Unless otherwise stated, structures depicted herein are also meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by the formulae of the present invention, as well as any wholly or partially equilibrated mixtures thereof. The present invention also includes the individual isomers of the compounds represented by the formulae above as mixtures with isomers thereof in which one or more chiral centers are inverted.

The present invention includes a salt or solvate of the compounds herein described, including combinations thereof such as a solvate of a salt. The compounds of the present invention may exist in solvated, for example hydrated, as well as unsolvated forms, and the present invention encompasses all such forms.

Typically, but not absolutely, the salts of the present invention are pharmaceutically acceptable salts. Salts encompassed within the term “pharmaceutically acceptable salts” refer to non-toxic salts of the compounds of this invention.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates. Representative salts are provided as described in U.S. Pat. No. 5,597,919 to Dull et al., U.S. Pat. No. 5,616,716 to Dull et al. and U.S. Pat. No. 5,663,356 to Ruecroft et al, each of which is herein incorporated by reference with regard to such salts.

As noted herein, the present invention includes specific representative compounds, which are identified herein with particularity. The compounds of this invention may be made by a variety of methods, including well-known standard synthetic methods. Illustrative general synthetic methods are set out below and then specific compounds of the invention are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts, Protecting Groups in Organic Synthesis, 3^{rd} Edition, John Wiley & Sons, New York (1999), incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention.

Those skilled in the art will recognize if a stereocenter exists. As noted hereinabove, the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be effected by any suitable method known in the art. See, for example, Stereochemistry of Organic Compounds (Wiley-Interscience, 1994), incorporated by reference with regard to stereochemistry.

The present invention also provides a method for the synthesis of compounds useful as intermediates in the preparation of compounds of the present invention along with methods for their preparation.

The compounds can be prepared according to the following methods using readily available starting materials and reagents. In these reactions, variants may be employed which are themselves known to those of ordinary skill in this art, but are not mentioned in greater detail.

### II. General Synthetic Methods

Compounds of the present invention include derivatives of both 2,6-diazabicyclo[3.2.1]octanes and 3,6-diazabicyclo[3.2.1]octanes. A method for the synthesis of suitably protected 3,6-diazabicyclo[3.2.1]octanes has been described in PCT WO 05/028477 to Basha et al., herein incorporated by reference with regard to such synthetic procedure. In this procedure, formalin and ammonium chloride are combined with cyclopentadiene, followed by reaction with di-tert-butyl dicarbonate, to afford 2-(tert-butoxycarbonyl)-2-azabicyclo[2.2.1]hept-5-ene. Sequential treatment with ozone and dimethylsulfide produces 1-(tert-butoxycarbonyl)-2,4-diformylpyrrolidine. Treatment of 1-(tert-butoxycarbonyl)-2,4-diformylpyrrolidine with benzylamine and sodium cyanoborohydride affords 6-(tert-butoxycarbonyl)-3-benzyl-3,6-diazabicyclo[3.2.1]octane. To produce mono-protected diazabicyclic amine compounds, either the benzyl group can be removed by hydrogenation or the tert-butoxycarbonyl group can be removed by treatment with strong acid, affording 6-(tert-butoxycarbonyl)-3,6-diazabicyclo[3.2.1]octane and 3-benzyl-3,6-diazabicyclo[3.2.1]octane respectively. Methods of separating the enantiomeric forms of 3,6-diazabicyclo[3.2.1]octanes are known to those of skill in the art of organic synthesis. Thus, resolution by formation of diastereomeric salts, using single enantiomer chiral acids, is possible, as well as resolution by formation of diastereomeric intermediates (for instance, as would be produced by the use of either (R)- or (S)-1-phenylethylamine in place of benzylamine in the reductive amination step) that can be separated by chromatographic means. Thus produced and suitably protected, these single enantiomer forms can be converted into compounds of the present invention.

Alternately, suitably protected single enantiomer 3,6-diazabicyclo[3.2.1]octanes can be made from single enantiomer starting materials. Thus, sequential treatment of commercially available (1R)-2-azabicyclo[2.2.1]hept-5-en-3-one or (1S)-2-azabicyclo[2.2.1]hept-5-en-3-one with lithium aluminum hydride and di-tert-butyl dicarbonate will generate (1R)-2-(tert-butoxycarbonyl)-2-azabicyclo[2.2.1]hept-5-ene and generate (1S)-2-(tert-butoxycarbonyl)-2-azabicyclo[2.2.1]hept-5-ene respectively. These single enantiomer intermediates can be transformed, as described above for the corresponding racemate, into the single enantiomers of 6-(tert-butoxycarbonyl)-3,6-diazabicyclo[3.2.1]octane and 3-benzyl-3,6-diazabicyclo[3.2.1]octane. In a different approach, the single enantiomer tert-butyl 2,4-diformylpyrrolidin-1-carboxylates can be converted into the single enantiomer 3,6-diazabicyclo[3.2.1]octanes by reduction of the formyl groups to the corresponding alcohols, followed by formation of the di-mesylate derivatives and cyclization with ammonia and cuprous iodide. This produces the enantiomeric 6-(tert-butoxycarbonyl)-3,6-diazabicyclo[3.2.1]octanes directly, without having to remove a benzyl protecting group. The enantiomeric tert-butyl 3,6-diazabicyclo[3.2.1]octane-6-carboxylates are suitable intermediates for conversion into compounds of the present invention.

For compounds of the present invention, the 3,6-diazabicyclo[3.2.1]octane scaffold was prepared as illustrated in Scheme 1 using a modified version of the methods described above. Treatment of commercially available 2-azabicyclo[2.2.1]hept-5-en-3-one (1) with lithium aluminum hydride followed by reaction with di-tert-butyl dicarbonate gave 2-(tert-butoxycarbonyl)-2-azabicyclo[2.2.1]hept-5-ene (2). Treatment of this compound with ozone followed by reduction with sodium borohydride gave 1-(tert-butoxycarbonyl)-2,4-bis(hydroxymethyl)pyrrolidine (3). Reaction of this compound with methanesulfonyl chloride afforded 1-(tert-butoxycarbonyl)-2,4-bis((methylsulfonyloxy)methyl)pyrrolidine (4) (Ms=methanesulfonyl), which was reacted in a sealed tube with copper iodide and ammonium hydroxide to yield 6-(tert-butoxycarbonyl)-3,6-diazabicyclo[3.2.1]octane (5).

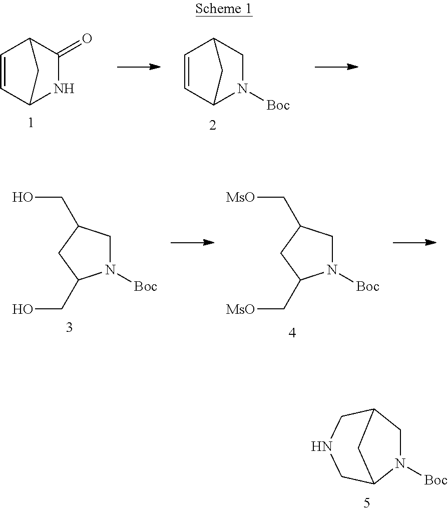

Compound 5 can undergo a protection/deprotection sequence to give 3-(trifluoroacetyl)-3,6-diazabicyclo[3.2.1]octane 6 (Scheme 2) by treatment of 5 with trifluoroacetic anhydride, followed by removal of the tert-butoxycarbonyl protecting group by treatment with trifluoroacetic acid. Such methods for installation and removal of the tert-butoxycarbonyl and trifluoroacetate amine protecting groups which are well known by those skilled in the art and are described in T. W. Greene and P. G. M. Wuts, Protective Groups in Organic Synthesis, 3^{rd} Edition, John Wiley & Sons, New York (1999).

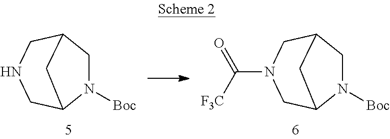

The compounds of the present invention can be prepared via the coupling of mono-protected diazabicycle (5 or 6) with a suitably functionalized acid chloride, chioroformate, sulfonylchloride, isocyanate, isothiocyanate, or other reactive derivative. Such compounds may be available commercially or prepared by methods that are well known to those skilled in the art and are described in, for example, in M. B. Smith and J. March, March's Advanced Organic. Chemistry: Reactions, Mechanisms and Structure, Sixth Edition, John Wiley & Sons, New York (2007), herein incorporated by reference with regard to such procedure. After coupling, removal of the boc or TFA protecting group can be achieved by treatment with acid or base, respectively, to afford the compounds of the present invention. Other compounds of the present invention can be synthesized by alkylation of the remaining basic nitrogen with an activated alkyl compound such as an alkyl halide.

Methods for the synthesis of a suitably protected 2,6-diazabicyclo[3.2.1]octanes can vary. One such method is described in PCT WO 05/028477 to Basha et al., which is herein incorporated by reference with regard to such synthetic teaching, in which benzyl 5-oxo-2-azabicyclo[2.2.1]heptane-2-carboxylate (prepared according to the procedure described by Carroll, et al., J. Med. Chem. 35: 2184 (1992), which is herein incorporated by reference with regard to such synthetic teaching), is converted into its oxime derivative, which is then stirred with trimethylsilylpolyphosphate to effect ring expansion, giving benzyl 3-oxo-2,6-diazabicyclo[3.2.1]octane-6-carboxylate. Sequential treatment with borane-methyl sulfide complex and n-propylamine gives the mono-protected diazabicyclic product, benzyl 2,6-diazabicyclo[3.2.1]octane-6-carboxylate. Protection of the free 2-position amine with di-tert-butyl dicarbonate, followed by hydrogenolysis of the benzyloxycarbonyl protecting group, gives another mono-protected diazabicycle, tert-butyl 2,6-diazabicyclo[3.2.1]octane-2-carboxylate. Compounds of the present invention can be prepared via coupling either benzyl 2,6-diazabicyclo[3.2.1]octane-6-carboxylate or tert-butyl 2,6-diazabicyclo[3.2.1]octane-2-carboxylate with a suitably functionalized acid chloride, chloroformate, sulfonylchloride, isocyanate, isothiocyanate, or other reactive derivative, followed by removal of the protecting group.

Methods of separating the enantiomeric forms of 2,6-diazabicyclo[3.2.1]octanes are known to those of skill in the art of organic synthesis. Thus, resolution by formation of diastereomeric salts, using single enantiomer chiral acids, is possible, as well as resolution by formation of diastereomeric intermediates that can be separated by chromatographic means. Thus produced and suitably protected, these single enantiomer forms can be converted into compounds of the present invention.

Those skilled in the art of organic synthesis will appreciate that there exist multiple means of producing compounds of the present invention which are labeled with a radioisotope appropriate to various diagnostic uses. Thus, condensation of a ¹¹C- or ¹⁸F-labeled reactive derivative with either compound 5 or compound 6 followed by removal of the protecting group as described above will produce a compound suitable for use in positron emission tomography. Further derivatization of this compound is possible as described above by alkylation of the remaining basic nitrogen with an activated alkyl compound.

### III. Pharmaceutical Compositions

The pharmaceutical compositions of the present invention include the salts described herein, in the pure state or in the form of a composition in which the compounds are combined with any other pharmaceutically compatible product, which can be inert or physiologically active. The resulting pharmaceutical compositions can be used to prevent a condition or disorder in a subject susceptible to such a condition or disorder, and/or to treat a subject suffering from the condition or disorder. The pharmaceutical compositions described herein include one or more compounds of Formula I and/or pharmaceutically acceptable salts thereof.

The manner in which the compounds are administered can vary. The compositions are preferably administered orally (e.g., in liquid form within a solvent such as an aqueous or non-aqueous liquid, or within a solid carrier). Preferred compositions for oral administration include pills, tablets, capsules, caplets, syrups, and solutions, including hard gelatin capsules and time-release capsules. Standard excipients include binders, fillers, colorants, solubilizers and the like. Compositions can be formulated in unit dose form, or in multiple or subunit doses. Preferred compositions are in liquid or semisolid form. Compositions including a liquid pharmaceutically inert carrier such as water or other pharmaceutically compatible liquids or semisolids can be used. The use of such liquids and semisolids is well known to those of skill in the art.

The compositions can also be administered via injection, i.e., intravenously, intramuscularly, subcutaneously, intraperitoneally, intraarterially, intrathecally; and intracerebroventricularly. Intravenous administration is the preferred method of injection. Suitable carriers for injection are well known to those of skill in the art and include 5% dextrose solutions, saline, and phosphate-buffered saline. The compounds can also be administered as an infusion or injection (e.g., as a suspension or as an emulsion in a pharmaceutically acceptable liquid or mixture of liquids).

The formulations can also be administered using other means, for example, rectal administration. Formulations useful for rectal administration, such as suppositories, are well known to those of skill in the art. The compounds can also be administered by inhalation (e.g., in the form of an aerosol either nasally or using delivery articles of the type set forth in U.S. Pat. No. 4,922,901 to Brooks et al., the disclosure of which is incorporated herein in its entirety); topically (e.g., in lotion form); transdermally (e.g., using a transdermal patch) or iontophoretically; or by sublingual or buccal administration. Although it is possible to administer the compounds in the form of a bulk active chemical, it is preferred to present each compound in the form of a pharmaceutical composition or formulation for efficient and effective administration.

Exemplary methods for administering such compounds will be apparent to the skilled artisan. The usefulness of these formulations can depend on the particular composition used and the particular subject receiving the treatment. These formulations can contain a liquid carrier that can be oily, aqueous, emulsified or contain certain solvents suitable to the mode of administration.

The compositions can be administered intermittently or at a gradual, continuous, constant or controlled rate to a warm-blooded animal (e.g., a mammal such as a mouse, rat, cat, rabbit, dog, pig, cow, or monkey), but advantageously are administered to a human being. In addition, the time of day and the number of times per day that the pharmaceutical formulation is administered can vary. Other suitable methods for administering the compounds of the present invention are described in U.S. Pat. No. 5,604,231 to Smith et al., the contents of which are hereby incorporated by reference.

In an embodiment of the present invention and as will be appreciated by those skilled in the art, the compound of the present invention may be administered in combination with other therapeutic compounds. For example, a compound of this invention can be used in combination with other NNR ligands (such as varenicline), antioxidants (such as free radical scavenging agents), antibacterial agents (such as penicillin antibiotics), antiviral agents (such as nucleoside analogs, like zidovudine and acyclovir), anticoagulants (such as warfarin), anti-inflammatory agents (such as NSAIDs), anti-pyretics, analgesics, anesthetics (such as used in surgery), acetylcholinesterase inhibitors (such as donepezil and galantamine), antipsychotics (such as haloperidol, clozapine, olanzapine, and quetiapine), immuno-suppressants (such as cyclosporin and methotrexate), neuroprotective agents, steroids (such as steroid hormones), corticosteroids (such as dexamethasone, predisone, and hydrocortisone), vitamins, minerals, nutraceuticals, anti-depressants (such as imipramine, fluoxetine, paroxetine, escitalopram, sertraline, venlafaxine, and duloxetine), anxiolytics (such as alprazolam and buspirone), anticonvulsants (such as phenytoin and gabapentin), vasodilators (such as prazosin and sildenafil), mood stabilizers (such as valproate and aripiprazole), anti-cancer drugs (such as anti-proliferatives), antihypertensive agents (such as atenolol, clonidine, amlopidine, verapamil, and olmesartan), laxatives, stool softeners, diuretics (such as furosemide), anti-spasmotics (such as dicyclomine), anti-dyskinetic agents, and anti-ulcer medications (such as esomeprazole).

The compounds of the present invention may be employed alone or in combination with other therapeutic agents, including other compounds of the present invention. Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration in combination of a compound of the formulae of the present invention including salts or solvates thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The compounds of the present invention may be used in the treatment of a variety of disorders and conditions and, as such, the compounds of the present invention may be used in combination with a variety of other suitable therapeutic agents useful in the treatment or prophylaxis of those disorders or conditions.

The following examples are provided to illustrate the present invention, and should not be construed as limiting thereof. In these examples, all parts and percentages are by weight, unless otherwise noted.

The appropriate dose of the compound is that amount effective to prevent occurrence of the symptoms of the disorder or to treat some symptoms of the disorder from which the patient suffers. By “effective amount”, “therapeutic amount” or “effective dose” is meant that amount sufficient to elicit the desired pharmacological or therapeutic effects, thus resulting in effective prevention or treatment of the disorder.

When treating a CNS disorder, an effective amount of compound is an amount sufficient to pass across the blood-brain barrier of the subject, to bind to relevant receptor sites in the brain of the subject and to modulate the activity of relevant NNR subtypes (e.g., provide neurotransmitter secretion, thus resulting in effective prevention or treatment of the disorder). Prevention of the disorder is manifested by delaying the onset of the symptoms of the disorder. Treatment of the disorder is manifested by a decrease in the symptoms associated with the disorder or an amelioration of the recurrence of the symptoms of the disorder. Preferably, the effective amount is sufficient to obtain the desired result, but insufficient to cause appreciable side effects.

The effective dose can vary, depending upon factors such as the condition of the patient, the severity of the symptoms of the disorder, and the manner in which the pharmaceutical composition is administered. For human patients, the effective dose of typical compounds generally requires administering the compound in an amount sufficient to modulate the activity of relevant NNRs, but the amount should be insufficient to induce effects on skeletal muscles and ganglia to any significant degree. The effective dose of compounds will of course differ from patient to patient, but in general includes amounts starting where CNS effects or other desired therapeutic effects occur but below the amount where muscular effects are observed.

The compounds described herein, when employed in effective amounts in accordance with the methods described herein, can provide some degree of prevention of the progression of, ameliorate symptoms of, and ameliorate to some degree of the recurrence of CNS or other disorders. The effective amounts of those compounds are typically below the threshold concentration required to elicit any appreciable side effects, for example those effects relating to skeletal muscle or ganglia. The compounds can be administered in a therapeutic window in which certain CNS and other disorders are treated and certain side effects are avoided. Ideally, the effective dose of the compounds described herein is sufficient to provide the desired effects upon the disorder but is insufficient (i.e., is not at a high enough level) to provide undesirable side effects. Preferably, the compounds are administered at a dosage effective for treating the CNS or other disorders but less than ⅕, and often less than 1/10, the amount required to elicit certain side effects to any significant degree.

Most preferably, effective doses are at very low concentrations, where maximal effects are observed to occur, with a minimum of side effects. An effective dose of such compounds may require administering the compound in an amount of less than 5 mg/kg of patient weight. The compounds of the present invention may be administered in an amount from less than about 1 mg/kg patent weight and usually less than about 100 μg/kg of patient weight, but may be between about 10 μg to less than 100 μg/kg of patient weight. The foregoing doses typically represent that amount administered as a single dose, or as one or more doses administered over a 24-hour period.

For human patients, an effective dose of typical compounds generally requires administering the compound in an amount of at least about 1, often at least about 10, and frequently at least about 100 mg/24 hr/patient. For human patients, an effective dose of typical compounds requires administering the compound which generally does not exceed about 500, often does not exceed about 400, and frequently does not exceed about 300 mg/24 hr/patient. In addition, the compositions may be advantageously administered at an effective dose such that the concentration of the compound within the plasma of the patient normally does not exceed 50 ng/mL, often does not exceed 30 ng/mL, and frequently does not exceed 10 ng/mL.

### IV. Method of Using Pharmaceutical Compositions

The compounds of the present invention can be used for the prevention or treatment of various conditions or disorders for which other types of nicotinic compounds have been proposed or are shown to be useful as therapeutics, such as CNS disorders, inflammation, inflammatory response associated with bacterial and/or viral infection, pain, metabolic syndrome, autoimmune disorders, addictions, obesity or other disorders described in further detail herein. This compound can also be used as a diagnostic agent in receptor binding studies (in vitro and in vivo). Such therapeutic and other teachings are described, for example, in references previously listed herein, including Williams et al., Drug News Perspec. 7(4): 205 (1994), Arneric et al., CNS Drug Rev. 1(1): 1-26 (1995), Arneric et al., Exp. Opin. Invest. Drugs 5(1): 79-100 (1996), Bencherif et al., J. Pharmacol. Exp. Ther. 279: 1413 (1996), Lippiello et al., J. Pharmacol. Exp. Ther. 279: 1422 (1996), Damaj et al., J. Pharmacol. Exp. Ther. 291: 390 (1999); Chiari et al., Anesthesiology 91: 1447 (1999), Lavand'homme and Eisenbach, Anesthesiology 91: 1455 (1999), Holladay et al., J. Med. Chem. 40(28): 4169-94 (1997), Bannon et al., Science 279: 77 (1998), PCT WO 94/08992, PCT WO 96/31475, PCT WO 96/40682, and U.S. Pat. No. 5,583,140 to Bencherif et al., U.S. Pat. No. 5,597,919 to Dull et al., U.S. Pat. No. 5,604,231 to Smith et al. and U.S. Pat. No. 5,852,041 to Cosford et al.

### CNS Disorders

The compounds and their pharmaceutical compositions are useful in the treatment or prevention of a variety of CNS disorders, including neurodegenerative disorders, neuropsychiatric disorders, neurologic disorders, and addictions. The compounds and their pharmaceutical compositions can be used to treat or prevent cognitive deficits and dysfunctions, age-related and otherwise; attentional disorders and dementias, including those due to infectious agents or metabolic disturbances; to provide neuroprotection; to treat convulsions and multiple cerebral infarcts; to treat mood disorders, compulsions and addictive behaviors; to provide analgesia; to control inflammation, such as mediated by cytokines and nuclear factor kappa B; to treat inflammatory disorders; to provide pain relief; and to treat infections, as anti-infectious agents for treating bacterial, fungal, and viral infections. Among the disorders, diseases and conditions that the compounds and pharmaceutical compositions of the present invention can be used to treat or prevent are: age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, central ischemia, peripheral ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism including Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, tardive dyskinesia, hyperkinesia, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), and chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, conduct disorder, oppositional defiant disorder, Tourette's syndrome, autism, drug and alcohol addiction, tobacco addiction, obesity, cachexia, psoriasis, lupus, acute cholangitis, aphthous stomatitis, ulcers, asthma, ulcerative colitis, inflammatory bowel disease, Crohn's disease, irritable bowel syndrome, spastic dystonia, diarrhea, constipation, pouchitis, viral pneumonitis, arthritis, including, rheumatoid arthritis and osteoarthritis, endotoxaemia, sepsis, atherosclerosis, idiopathic pulmonary fibrosis, acute pain, chronic pain, neuropathies, urinary incontinence, diabetes, sexual dysfunction, neoplasias, and preeclampsia.

Cognitive impairments or dysfunctions may be associated with psychiatric disorders or conditions, such as schizophrenia and other psychotic disorders, including but not limited to psychotic disorder, schizophreniform disorder, schizoaffective disorder, delusional disorder, brief psychotic disorder, shared psychotic disorder, and psychotic disorders due to a general medical conditions, dementias and other cognitive disorders, including but not limited to mild cognitive impairment, pre-senile dementia, Alzheimer's disease, senile dementia, dementia of the Alzheimer's type, age-related memory impairment, Lewy body dementia, vascular dementia, AIDS dementia complex, dyslexia, Parkinsonism including Parkinson's disease, cognitive impairment and dementia of Parkinson's Disease, cognitive impairment of multiple sclerosis, cognitive impairment caused by traumatic brain injury, dementias due to other general medical conditions, anxiety disorders, including but not limited to panic disorder without agoraphobia, panic disorder with agoraphobia, agoraphobia without history of panic disorder, specific phobia, social phobia, obsessive-compulsive disorder, post-traumatic stress disorder, acute stress disorder, generalized anxiety disorder and generalized anxiety disorder due to a general medical condition, mood disorders, including but not limited to major depressive disorder, dysthymic disorder, bipolar depression, bipolar mania, bipolar I disorder, depression associated with manic, depressive or mixed episodes, bipolar II disorder, cyclothymic disorder, and mood disorders due to general medical conditions, sleep disorders, including but not limited to dyssomnia disorders, primary insomnia, primary hypersomnia, narcolepsy, parasomnia disorders, nightmare disorder, sleep terror disorder and sleepwalking disorder, mental retardation, learning disorders, motor skills disorders, communication disorders, pervasive developmental disorders, attention-deficit and disruptive behavior disorders, attention deficit disorder, attention deficit hyperactivity disorder, feeding and eating disorders of infancy, childhood, or adults, tic disorders, elimination disorders, substance-related disorders, including but not limited to substance dependence, substance abuse, substance intoxication, substance withdrawal, alcohol-related disorders, amphetamine or amphetamine-like-related disorders, caffeine-related disorders, cannabis-related disorders, cocaine-related disorders, hallucinogen-related disorders, inhalant-related disorders, nicotine-related disorders, opioid-related disorders, phencyclidine or phencyclidine-like-related disorders, and sedative-, hypnotic- or anxiolytic-related disorders, personality disorders, including but not limited to obsessive-compulsive personality disorder and impulse-control disorders.

Cognitive performance may be assessed with a validated cognitive scale, such as, for example, the cognitive subscale of the Alzheimer's Disease Assessment Scale (ADAS-cog). One measure of the effectiveness of the compounds of the present invention in improving cognition may include measuring a patient's degree of change according to such a scale.

Regarding compulsions and addictive behaviors, the compounds of the present invention may be used as a therapy for nicotine addiction and for other brain-reward disorders, such as substance abuse including alcohol addiction, illicit and prescription drug addiction, eating disorders, including obesity, and behavioral addictions, such as gambling, or other similar behavioral manifestations of addiction.

The above conditions and disorders are discussed in further detail, for example, in the American Psychiatric Association: Diagnostic and Statistical Manual of Mental Disorders, Fourth Edition, Text Revision, Washington, D.C., American Psychiatric Association, 2000. This Manual may also be referred to for greater detail on the symptoms and diagnostic features associated with substance use, abuse, and dependence.

Preferably, the treatment or prevention of diseases, disorders and conditions occurs without appreciable adverse side effects, including, for example, significant increases in blood pressure and heart rate, significant negative effects upon the gastro-intestinal tract, and significant effects upon skeletal muscle.

The compounds of the present invention, when employed in effective amounts, are believed to modulate the activity of the α4β2 and α7 NNRs without appreciable interaction with the nicotinic subtypes that characterize the human ganglia, as demonstrated by a lack of the ability to elicit nicotinic function in adrenal chromaffin tissue, or skeletal muscle, further demonstrated by a lack of the ability to elicit nicotinic function in cell preparations expressing muscle-type nicotinic receptors. Thus, these compounds are believed capable of treating or preventing diseases, disorders and conditions without eliciting significant side effects associated activity at ganglionic and neuromuscular sites. Thus, administration of the compounds is believed to provide a therapeutic window in which treatment of certain diseases, disorders and conditions is provided, and certain side effects are avoided. That is, an effective dose of the compound is believed sufficient to provide the desired effects upon the disease, disorder or condition, but is believed insufficient, namely is not at a high enough level, to provide undesirable side effects.

Thus, the present invention provides the use of a compound of the present invention, or a pharmaceutically acceptable salt thereof, for use in therapy, such as a therapy described above.

In yet another aspect the present invention provides the use of a compound of the present invention, or a pharmaceutically acceptable salt thereof, in the manufacture of a medicament for use in the treatment of a CNS disorder, such as a disorder, disease or condition described hereinabove.

### Inflammation

The nervous system, primarily through the vagus nerve, is known to regulate the magnitude of the innate immune response by inhibiting the release of macrophage tumor necrosis factor (TNF). This physiological mechanism is known as the “cholinergic anti-inflammatory pathway” (see, for example, Tracey, “The Inflammatory Reflex,” Nature 420: 853-9 (2002)). Excessive inflammation and tumor necrosis factor synthesis cause morbidity and even mortality in a variety of diseases. These diseases include, but are not limited to, endotoxemia, rheumatoid arthritis, osteoarthritis, psoriasis, asthma, atherosclerosis, idiopathic pulmonary fibrosis, and inflammatory bowel disease.

Inflammatory conditions that can be treated or prevented by administering the compounds described herein include, but are not limited to, chronic and acute inflammation, psoriasis, endotoxemia, gout, acute pseudogout, acute gouty arthritis, arthritis, rheumatoid arthritis, osteoarthritis, allograft rejection, chronic transplant rejection, asthma, atherosclerosis, mononuclear-phagocyte dependent lung injury, idiopathic pulmonary fibrosis, atopic dermatitis, chronic obstructive pulmonary disease, adult respiratory distress syndrome, acute chest syndrome in sickle cell disease, inflammatory bowel disease, Crohn's disease, ulcerative colitis, acute cholangitis, aphteous stomatitis, pouchitis, glomerulonephritis, lupus nephritis, thrombosis, and graft vs. host reaction.

Inflammatory Response Associated with Bacterial and/or Viral Infection

Many bacterial and/or viral infections are associated with side effects brought on by the formation of toxins, and the body's natural response to the bacteria or virus and/or the toxins. As discussed above, the body's response to infection often involves generating a significant amount of TNF and/or other cytokines. The over-expression of these cytokines can result in significant injury, such as septic shock (when the bacteria is sepsis), endotoxic shock, urosepsis and toxic shock syndrome.

Cytokine expression is mediated by NNRs, and can be inhibited by administering agonists or partial agonists of these receptors. Those compounds described herein that are agonists or partial agonists of these receptors can therefore be used to minimize the inflammatory response associated with bacterial infection, as well as viral and fungal infections. Examples of such bacterial infections include anthrax, botulism, and sepsis. Some of these compounds may also have antimicrobial properties.

These compounds can also be used as adjunct therapy in combination with existing therapies to manage bacterial, viral and fungal infections, such as antibiotics, antivirals and antifungals. Antitoxins can also be used to bind to toxins produced by the infectious agents and allow the bound toxins to pass through the body without generating an inflammatory response. Examples of antitoxins are disclosed, for example, in U.S. Pat. No. 6,310,043 to Bundle et al. Other agents effective against bacterial and other toxins can be effective and their therapeutic effect can be complemented by co-administration with the compounds described herein.

### Pain

The compounds can be administered to treat and/or prevent pain, including acute, neurologic, inflammatory, neuropathic and chronic pain. The compounds can be used in conjunction with opiates to minimize the likelihood of opiate addiction (e.g., morphine sparing therapy). The analgesic activity of compounds described herein can be demonstrated in models of persistent inflammatory pain and of neuropathic pain, performed as described in U.S. Published Patent Application No. 20010056084 A1 (Allgeier et al.) (e.g., mechanical hyperalgesia in the complete Freund's adjuvant rat model of inflammatory pain and mechanical hyperalgesia in the mouse partial sciatic nerve ligation model of neuropathic pain).

The analgesic effect is suitable for treating pain of various genesis or etiology, in particular in treating inflammatory pain and associated hyperalgesia, neuropathic pain and associated hyperalgesia, chronic pain (e.g., severe chronic pain, post-operative pain and pain associated with various conditions including cancer, angina, renal or biliary colic, menstruation, migraine, and gout). Inflammatory pain may be of diverse genesis, including arthritis and rheumatoid disease, teno-synovitis and vasculitis. Neuropathic pain includes trigeminal or herpetic neuralgia, diabetic neuropathy pain, causalgia, low back pain and deafferentation syndromes such as brachial plexus avulsion.

### Neovascularization

The α7 NNR is associated with neovascularization. Inhibition of neovascularization, for example, by administering antagonists (or at certain dosages, partial agonists) of the α7 NNR can treat or prevent conditions characterized by undesirable neovascularization or angiogenesis. Such conditions can include those characterized by inflammatory angiogenesis and/or ischemia-induced angiogenesis. Neovascularization associated with tumor growth can also be inhibited by administering those compounds described herein that function as antagonists or partial agonists of α7 NNR.

Specific antagonism of α7 NNR-specific activity reduces the angiogenic response to inflammation, ischemia, and neoplasia. Guidance regarding appropriate animal model systems for evaluating the compounds described herein can be found, for example, in Heeschen, C. et al., “A novel angiogenic pathway mediated by non-neuronal nicotinic acetylcholine receptors,” J. Clin. Invest. 110(4):527-36 (2002).

Representative tumor types that can be treated using the compounds described herein include NSCLC, ovarian cancer, pancreatic cancer, breast carcinoma, colon carcinoma, rectum carcinoma, lung carcinoma, oropharynx carcinoma, hypopharynx carcinoma, esophagus carcinoma, stomach carcinoma, pancreas carcinoma, liver carcinoma, gallbladder carcinoma, bile duct carcinoma, small intestine carcinoma, urinary tract carcinoma, kidney carcinoma, bladder carcinoma, urothelium carcinoma, female genital tract carcinoma, cervix carcinoma, uterus carcinoma, ovarian carcinoma, choriocarcinoma, gestational trophoblastic disease, male genital tract carcinoma, prostate carcinoma, seminal vesicles carcinoma, testes carcinoma, germ cell tumors, endocrine gland carcinoma, thyroid carcinoma, adrenal carcinoma, pituitary gland carcinoma, skin carcinoma, hemangiomas, melanomas, sarcomas, bone and soft tissue sarcoma, Kaposi's sarcoma, tumors of the brain, tumors of the nerves, tumors of the eyes, tumors of the meninges, astrocytomas, gliomas, glioblastomas, retinoblastomas, neuromas, neuroblastomas, Schwannomas, meningiomas, solid tumors arising from hematopoietic malignancies (such as leukemias, chloromas, plasmacytomas and the plaques and tumors of mycosis fungoides and cutaneous T-cell lymphoma/leukemia), and solid tumors arising from lymphomas.

The compounds can also be administered in conjunction with other forms of anti-cancer treatment, including co-administration with antineoplastic antitumor agents such as cis-platin, adriamycin, daunomycin, and the like, and/or anti-VEGF (vascular endothelial growth factor) agents, as such are known in the art.

The compounds can be administered in such a manner that they are targeted to the tumor site. For example, the compounds can be administered in microspheres, microparticles or liposomes conjugated to various antibodies that direct the microparticles to the tumor. Additionally, the compounds can be present in microspheres, microparticles or liposomes that are appropriately sized to pass through the arteries and veins, but lodge in capillary beds surrounding tumors and administer the compounds locally to the tumor. Such drug delivery devices are known in the art.

### Other Disorders

In addition to treating CNS disorders, inflammation, and neovascularization, and pain, the compounds of the present invention can be also used to prevent or treat certain other conditions, diseases, and disorders in which NNRs play a role. Examples include autoimmune disorders such as Lupus, disorders associated with cytokine release, cachexia secondary to infection (e.g., as occurs in AIDS, AIDS related complex and neoplasia), obesity, pemphitis, urinary incontinence, retinal diseases, infenctious diseases, myasthenia, Eaton-Lambert syndrome, hypertension, preeclampsia, osteoporosis, vasoconstriction, vasodilatation, cardiac arrhythmias, type I diabetes, bulimia, anorexia as well as those indications set forth in published PCT application WO 98/25619. The compounds of this invention can also be administered to treat convulsions such as those that are symptomatic of epilepsy, and to treat conditions such as syphillis and Creutzfeld-Jakob disease.

### Diagnostic Uses

The compounds can be used in diagnostic compositions, such as probes, particularly when they are modified to include appropriate labels. The probes can be used, for example, to determine the relative number and/or function of specific receptors, particularly the α4β2 and α7 receptor subtypes. For this purpose the compounds of the present invention most preferably are labeled with a radioactive isotopic moiety such as ¹¹C, ¹⁸F, ⁷⁶Br, ¹²³I or ¹²⁵I.

The administered compounds can be detected using known detection methods appropriate for the label used. Examples of detection methods include position emission topography (PET) and single-photon emission computed tomography (SPECT). The radiolabels described above are useful in PET (e.g., ¹¹C, ¹⁸F or ⁷⁶Br) and SPECT (e.g., ¹²³I) imaging, with half-lives of about 20.4 minutes for ¹¹C, about 109 minutes for ¹⁸F, about 13 hours for ¹²³I, and about 16 hours for ⁷⁶Br. A high specific activity is desired to visualize the selected receptor subtypes at non-saturating concentrations. The administered doses typically are below the toxic range and provide high contrast images. The compounds are expected to be capable of administration in non-toxic levels. Determination of dose is carried out in a manner known to one skilled in the art of radiolabel imaging. See, for example, U.S. Pat. No. 5,969,144 to London et al.

The compounds can be administered using known techniques. See, for example, U.S. Pat. No. 5,969,144 to London et al., as noted. The compounds can be administered in formulation compositions that incorporate other ingredients, such as those types of ingredients that are useful in formulating a diagnostic composition. Compounds useful in accordance with carrying out the present invention most preferably are employed in forms of high purity. See, U.S. Pat. No. 5,853,696 to Elmalch et al.

After the compounds are administered to a subject (e.g., a human subject), the presence of that compound within the subject can be imaged and quantified by appropriate techniques in order to indicate the presence, quantity, and functionality of selected NNR subtypes. In addition to humans, the compounds can also be administered to animals, such as mice, rats, dogs, and monkeys. SPECT and PET imaging can be carried out using any appropriate technique and apparatus. See Villemagne et al., In: Arneric et al. (Eds.) Neuronal Nicotinic Receptors: Pharmacology and Therapeutic Opportunities, 235-250 (1998) and U.S. Pat. No. 5,853,696 to Elmalch et al., each herein incporated by reference, for a disclosure of representative imaging techniques.

The radiolabeled compounds bind with high affinity to selective NNR subtypes (e.g., α4β2, α7) and preferably exhibit negligible non-specific binding to other nicotinic cholinergic receptor subtypes (e.g., those receptor subtypes associated with muscle and ganglia). As such, the compounds can be used as agents for noninvasive imaging of nicotinic cholinergic receptor subtypes within the body of a subject, particularly within the brain for diagnosis associated with a variety of CNS diseases and disorders.

In one aspect, the diagnostic compositions can be used in a method to diagnose disease in a subject, such as a human patient. The method involves administering to that patient a detectably labeled compound as described herein, and detecting the binding of that compound to selected NNR subtypes (e.g., α4β2 and α7 receptor subtypes). Those skilled in the art of using diagnostic tools, such as PET and SPECT, can use the radiolabeled compounds described herein to diagnose a wide variety of conditions and disorders, including conditions and disorders associated with dysfunction of the central and autonomic nervous systems. Such disorders include a wide variety of CNS diseases and disorders, including Alzheimer's disease, Parkinson's disease, and schizophrenia. These and other representative diseases and disorders that can be evaluated include those that are set forth in U.S. Pat. No. 5,952,339 to Bencherif et al.

In another aspect, the diagnostic compositions can be used in a method to monitor selective nicotinic receptor subtypes of a subject, such as a human patient. The method involves administering a detectably labeled compound as described herein to that patient and detecting the binding of that compound to selected nicotinic receptor subtypes namely, the α4β2 and α7 receptor subtypes.

### Receptor Binding

The compounds of this invention can be used as reference ligands in binding assays for compounds which bind to NNR subtypes, particularly the α4β2 and α7 receptor subtypes. For this purpose the compounds of this invention are preferably labeled with a radioactive isotopic moiety such as ³H, or ¹⁴C. Examples of such binding assays are described in detail below.

### SYNTHETIC EXAMPLES

The following examples are provided to illustrate the present invention, and should not be construed as limiting thereof. In these examples, all parts and percentages are by weight, unless otherwise noted.

### Example 1

### Synthesis of 6-(tert-butoxycarbonyl)-3,6-diazabicyclo[3.2.1]octane

The following general procedures can be employed using either racemic or single enantiomer starting materials, all of which are commercially available. The racemic synthesis is reported in detail here. Using similar procedures (1R,5S)-6-(tert-butoxycarbonyl)-3,6-diazabicyclo[3.2.1]octane was obtained in 35% overall yield from (1S,4R)-2-azabicyclo[2.2.1]hept-5-en-3-one (Aldrich Chemical), and (1S,5R)-6-(tert-butoxycarbonyl)-3,6-diazabicyclo[3.2.1]octane was obtained in 45% overall yield from (1R,4S)-2-azabicyclo[2.2.1]hept-5-en-3-one (Aldrich Chemical).

A solution of 2-azabicyclo[2.2.1]hept-5-en-3-one (5.0 g, 49 mmol) in dry tetrahydrofuran (THF) (100 mL) was added to a slurry of lithium aluminum hydride (1.8 g, 49 mmol) in dry THF (100 mL) at 0° C. The reaction mixture was heated at reflux for 3 h and then cooled to ambient temperature. Ether (100 mL) was added and the mixture was cooled and stirred at 0° C. as sodium hydroxide solution (5N, 20 mL) was slowly added to quench the reaction. The slurry was filtered through diatomaceous earth, and the filtrate was combined with di-tert-butyl dicarbonate (10.6 g, 48.6 mmol) and triethylamine (6.3 mL, 45 mmol). This mixture was stirred at ambient temperature for 12 h. The solvent was removed by rotary evaporation, and the residue was dissolved in dichloromethane (200 mL), washed with saturated aqueous ammonium chloride (200 mL), and dried over anhydrous magnesium sulfate. Evaporation of the dichloromethane left 9.4 g of 2-(tert-butoxycarbonyl)-2-azabicyclo[2.2.1]hept-5-ene as an oil.

2-(Tert-butoxycarbonyl)-2-azabicyclo[2.2.1]hept-5-ene was dissolved in 200 mL of dichloromethane-methanol (2:1), and the solution was cooled to −78° C. Ozone was passed through the solution until it turned blue and then for a further 10 min. Argon was bubbled through the solution to remove excess ozone (the solution turned colorless). This process (ozone, followed by argon) was repeated one more time to ensure complete formation of the ozonide. Sodium borohydride (3.7 g, 97 mmol) was carefully added to the reaction mixture at −78° C., and the resulting mixture stirred for 16 h, as the temperature of the reaction was gradually increased to ambient. Saturated ammonium chloride solution (100 mL) was added, and the mixture was stirred for an additional 1 h. The mixture was extracted with dichloromethane (2×150 mL), and the combined organic extracts were dried over anhydrous magnesium sulfate. The solvent was removed by rotary evaporation to give 1-(tert-butoxycarbonyl)-2,4-bis(hydroxymethyl)pyrrolidine, as a light yellow oil.

1-(Tert-butoxycarbonyl)-2,4-bis(hydroxymethyl)pyrrolidine was dissolved in 300 mL of dry dichloromethane and cooled to 0° C. Triethylamine (9.7 mL, 70 mmol) was added to the cooled solution, followed by a careful addition of methanesulfonyl chloride (5.4 mL, 70 mmol). The reaction was stirred at ambient temperature for 16 h. Saturated ammonium chloride solution (200 mL) was added, and the layers were separated. The aqueous layer was washed with dichloromethane (200 mL), and the combined organic layers were dried over anhydrous magnesium sulfate, filtered, and concentrated by evaporation of the volatiles. The residual oil, 1-(tert-butoxycarbonyl)-2,4-bis((methylsulfonyloxy)methyl)pyrrolidine, was placed in 200 mL pressure tubes (˜10 mmol maximum in each tube). Concentrated aqueous ammonium hydroxide (150 mL) and copper iodide (190 mg, 10 mol %) were added to each pressure tube. The tubes were sealed and heated at 100° C. for 16 h. The tubes were cooled to ambient temperature, and the reaction mixture was concentrated by rotary evaporation at 60° C. (bath temperature). The solid was dissolved in methanol and filtered through diatomaceous earth to remove copper salts. The solvent was removed by rotary evaporation, and the residue was purified using an Analogix IntelliFlash 280 system with a SF25-120g Si column, eluting with a methanol in chloroform gradient (0-50% methanol over 30 min). Evaporation of the solvent gave 6-(tert-butoxycarbonyl)-3,6-diazabicyclo[3.2.1]octane as a viscous oil (4.1 g, 40%).

### Example 2

### Synthesis of 3-(trifluoroacetyl)-3,6-diazabicyclo[3.2.1]octane

To a solution of 6-(tert-butoxycarbonyl)-3,6-diazabicyclo[3.2.1]octane (500 mg, 2.36 mmol in dichloromethane (20 mL) was added triethylamine (325 μL, 1 mol eq) at 0° C. followed by addition of trifluoroacetic anhydride (328 μL, 1 mol eq). The solution was warmed to room temperature, washed with aqueous sodium acetate (50 mM solution, 20 mL) and dichloromethane (2×20 mL). The combined organic extracts were dried over sodium sulfate and filtered. The crude material was purified, using the Analogix IntelliFlash 280 system with a SF15-12g Si column eluting with a dichloromethane to dichloromethane:ethyl acetate (1:1) gradient over 24 min, to give 3-(trifluoroacetyl)-6-(tert-butoxycarbonyl)-3,6-diazabicyclo[3.2.1]octane (400 mg, 92%) as an orange oil.

3-(triflouroacetyl)-6-(tert-butoxycarbonyl)-3,6-diazabicyclo[3.2.1]octane (400 mg, 1.30 mmol), was dissolved in dichloromethane to which a 0.5 mL of a 25% trifluoroacetic acid in dichloromethane solution was added. The reaction mixture was left to stir for 2 h, then washed with a saturated sodium bicarbonate solution (20 mL). The aqueous layer was extracted with dichloromethane. The combined organic extracts were dried over magnesium sulfate and purified, using the Analogix IntelliFlash 280 system with a SF15-12g Si column eluting with a chloroform to 90:9:1 chloroform:methanol:ammonium hydroxide gradient over 24 min, to give 3-(trifluoroacetyl)-3,6-diazabicyclo[3.2.1]octane (250 mg, 90%) as a yellow oil.

### Example 3

### Synthesis of (1S,5S)-3-(methoxycarbonyl)-3,6-diazabicyclo[3.2.1]octane

To a solution of (1R,5S)-6-(tert-butoxycarbonyl)-3,6-diazabicyclo[3.2.1]octane (60 mg, 0.28 mmol) in dichloromethane (5 mL) was added triethylamine (67 mL, 2 mol eq). The reaction was cooled to 0° C. and methyl chloroformate (21 mL, 26 mmol 1.1 mol eq) was added. The reaction mixture was stirred for 1 h, and the solvent was removed in vacuo. The residue was combined with dichloromethane and a 50mM aqueous sodium acetate solution and the reaction mixture was stirred for a further 10 min and subsequently passed through a phase separator. The organic phase was concentrated in vacuo, and the residue was dissolved in 3 mL of ethyl acetate and combined with 3 mL of a 3N hydrochloric acid in ethyl acetate solution. The reaction mixture was stirred for 2 h. The solvent was removed in vacuo at 60° C. The resultant residue was taken up in a 1:1 mixture of methanol:dichloromethane and passed through a Biotage SCX-2 column (cation exchange resin). The solvent was removed in vacuo and the residue was purified, using Analogix IntelliFlash 280 system with a SF10-4g Si column eluting with a chloroform to 90:9:1 chloroform:methanol:ammonium hydroxide gradient over 10 min, to give (1S,5S)-3-(methoxycarbonyl)-3,6-diazabicyclo[3.2.1]octane (10 mg, 25%) as a clear oil. ¹H NMR (CD₃OD, 300 MHz): δ 4.06 (m, 1H), 3.85 (m, 2H), 3.67 (s, 3H), 3.36 (m, 1H), 2.74-3.14 (m, 3H), 2.40 (m, 1H), 1.71 (m, 2H). MS (m/z): 171 (M+1).

### Example 4

### Synthesis of (1S,5S)-3-(cyclopropylcarbonyl)-3,6-diazabicyclo[3.2.1]octane

Cyclopropanecarboxylic acid (24 μL, 0.30 mmol) and o-benzotriazol-1-yl-N,N,N′,N′-tetramethyluronium hexafluorophosphate (155 mg, 0.41 mmol, 1.7 mol eq) were stirred in a reaction vial with 5 mL of dry dichloromethane. After 10 min (1R,5S)-6-(tert-butoxycarbonyl)-3,6-diazabicyclo[3.2.1]octane (50 mg, 0.24 mmol) in 2.5 mL of dichloromethane was added, and the reaction was stirred for 2 h. Saturated ammonium chloride (5 mL) was added and the reaction mixture was stirred for 10 min and subsequently passed through a phase separator. The solvent was removed for the organic phase in vacuo, and the residue was dissolved in 3 mL of ethyl acetate and combined with 3 mL of a 3N hydrochloric acid in ethyl acetate solution. The reaction mixture was stirred for 2 h before removing solvent in vacuo at 60° C. The resultant residue was taken up in a 1:1 mixture of methanol and dichloromethane and passed through a Biotage SCX-2 column (cation exchange resin). The solvent was removed in vacuo and the residue was purified, using Analogix IntelliFlash 280 system with a SF10-4g Si column eluting with a chloroform to 90:9:1 chloroform:methanol:ammonium hydroxide gradient over 8 min, to give (1S,5S)-3-(cyclopropylcarbonyl)-3,6-diazabicyclo[3.2.1]octane (18 mg, 42%) as a clear oil. ¹H NMR of HCl salt (CD₃OD, 400 MHz): δ 4.41 (m, 1H), 4.26 (m, 1H), 4.13 (m, 1H), 3.36 (m, 3H), 2.95 (m, 1H), 2.78 (m, 1H), 2.08 (3, 2H), 1.95 (bs, 1H), 0.87 (m, 4H). MS (m/z): 181 (M+1).

### Example 5

### Synthesis of (1S,5R)-6-(cyclopropylcarbonyl)-3,6-diazabicyclo[3.2.1]octane

Cyclopropanecarboxylic acid (24 μL, 0.30 mmol) and o-benzotriazol-1-yl-N,N,N′,N′-tetramethyluronium hexafluorophosphate (155 mg, 0.410 mmol, 1.70 mol eq) were stirred in a reaction vial with 5 mL of dry dichloromethane. After 10 min (1R,5R)-3-(trifluoroacetyl)-3,6-diazabicyclo[3.2.1]octane (50 mg, 0.24 mmol) in 2.5 mL of dichloromethane was added and the reaction was stirred for 2 h. Saturated ammonium chloride (5 mL) was added and the reaction mixture was stirred for 10 min and subsequently passed through a phase separator. The solvent was removed from the organic phase (in vacuo), and the residue was dissolved in 3 mL of methanol and combined with 3 mL of a 2N aqueous potassium bicarbonate. The reaction mixture was left to stir for 2 h at 60° C. and the solvent removed in vacuo at 60° C. The resultant residue was taken up in a 1:1 mixture of methanol and dichloromethane and passed through a Biotage SCX-2 column (cation exchange resin). The solvent was removed in vacuo and the residue was purified, using Analogix IntelliFlash 280 system with a SF10-4g Si column eluting with a chloroform to 90:9:1 chloroform:methanol:ammonium hydroxide gradient over 8 min, to give (1 S,5R)-6-(cyclopropylcarbonyl)-3,6-diazabicyclo[3.2.1]octane (15 mg, 35%) as a clear oil. MS (m/z): 181 (M+1).

### Example 6

### Synthesis of (1S,5S)-3-(methylsulfonyl)-3,6-diazabicyclo[3.2.1]octane

To a solution of (1R,5S)-6-(tert-butoxycarbonyl)-3,6-diazabicyclo[3.2.1]octane (50 mg, 0.24 mmol) in 5 mL of dichloromethane was added triethylamine (67 μL, 2 mol eq) and the solution was cooled to 0° C. Methanesulfonyl chloride (20 A, 26 mmol, 1.1 mol eq) and the reaction mixture was stirred for 1 h. The solvent was removed in vacuo and the residue was combined with dichloromethane and 50 mM aqueous sodium acetate. The reaction mixture was stirred for 10 min and passed through a phase separator. The organic phase was concentrated in vacuo, and the residue was dissolved in 3 mL of ethyl acetate and combined with 3 mL of a 3N hydrochloric acid in ethyl acetate solution. The reaction mixture was left to stir for 2 h before removing solvent in vacuo at 60° C. The resultant residue was taken up in a 1:1 mixture of methanol and dichloromethane and passed through a Biotage SCX-2 column (cation exchange resin). The solvent was removed in vacuo and the residue was purified, using Analogix IntelliFlash 280 system with a SF10-4g Si column eluting with a chloroform to 90:9:1 chloroform:methanol:ammonium hydroxide gradient over 10 min, to give (1S,5S)-3-(methylsulfonyl)-3,6-diazabicyclo[3.2.1]octane (12 mg, 26%) as a clear oil. MS (m/z): 191 (M+1).

### Example 7

### Synthesis of (1S,5S)-3-(N-allylcarbamoyl)-3,6-diazabicyclo[3.2.1]octane

To a solution of (1R,5S)-6-(tert-butoxycarbonyl)-3,6-diazabicyclo[3.2.1]octane (100 mg, 0.47 mmol) in 10 mL of dichloromethane was added triethylamine (134 μL, 0.94 mmol, 2 mol eq), and the reaction was cooled to 0° C. Allyl isocyanate (42 μL, 0.47 mmol, 1 mol eq) was added and the reaction solution was stirred for 2 h. The solvent was removed in vacuo and the residue was combined with dichloromethane and 50 mM aqueous sodium acetate solution. The reaction mixture was stirred for 10 min, then passed through a phase separator. The organic phase was concentrated in vacuo, and the residue was dissolved in 2.5 mL of ethyl acetate, to which 2.5 mL of a 3N hydrochloric acid in ethyl acetate solution was added. The reaction mixture was allowed to stir for 2 h and the solvent was removed in vacuo at 60° C. The resultant residue was dissolved in 1:1 methanol:dichloromethane and passed through a Biotage SCX-2 column (cation exchange resin). The solvent was removed in vacuo and the residue was purified, using Analogix IntelliFlash 280 system with a SF10-4g Si column eluting with a chloroform to 90:9:1 chloroform:methanol:ammonium hydroxide gradient over 10 min, (1 S,5S)-3-(N-allylcarbamoyl)-3,6-diazabicyclo[3.2.1]octane (15 mg, 16%) as a clear oil. MS (m/z): 196 (M+1).

The above illustrated amide coupling procedures were used as a basis to make the compounds shown in Table 1. Reagents and conditions will be readily apparent to those skilled in the art. In some cases, compounds were characterized by nuclear magnetic resonance (NMR) data. In other cases, compounds were structurally characterized by LCMS.

### VIII. Biological Assays

### Example 8

### Radioligand Binding at CNS nAChRs

α4β2 nAChR Subtype

Preparation of membranes from rat cortex: Rats (female, Sprague-Dawley), weighing 150-250 g, were maintained on a 12 h light/dark cycle and were allowed free access to water and food supplied by PMI Nutrition International, Inc. Animals were anesthetized with 70% CO₂, and then decapitated. Brains were removed and placed on an ice-cold platform. The cerebral cortex was removed and placed in 20 volumes (weight:volume) of ice-cold preparative buffer (137 mM NaCl, 10.7 mM KCl, 5.8 mM KH₂PO₄, 8 mM Na₂HPO₄, 20 mM HEPES (free acid), 5 mM iodoacetamide, 1.6 mM EDTA, pH 7.4); PMSF, dissolved in methanol to a final concentration of 100 μM, was added and the suspension was homogenized by Polytron. The homogenate was centrifuged at 18,000×g for 20 min at 4° C. and the resulting pellet was re-suspended in 20 volumes of ice-cold water. After 60 min incubation on ice, a new pellet was collected by centrifugation at 18,000×g for 20 min at 4° C. The final pellet was re-suspended in 10 volumes of buffer and stored at −20° C.

Preparation of membranes from SH-EP1/human α4β2 clonal cells: Cell pellets from 40 150 mm culture dishes were pooled, and homogenized by Polytron (Kinematica GmbH, Switzerland) in 20 milliliters of ice-cold preparative buffer. The homogenate was centrifuged at 48,000 g for 20 minutes at 4° C. The resulting pellet was re-suspended in 20 mL of ice-cold preparative buffer and stored at −20° C.

On the day of the assay, the frozen membranes were thawed and spun at 48,000×g for 20 min. The supernatant was decanted and discarded. The pellet was resuspended in Dulbecco's phosphate buffered saline (PBS, Life Technologies) pH 7.4 and homogenized with the Polytron for 6 seconds. Protein concentrations were determined using a Pierce BCA Protein Assay Kit, with bovine serum albumin as the standard (Pierce Chemical Company, Rockford, Ill.).

Assay: Membrane preparations (approximately 50 μg for human and 200-300 μg protein for rat α4β2) were incubated in PBS (50 μL and 100 μL respectively) in the presence of competitor compound (0.01 nM to 100 μM) and 5 nM [³H]nicotine for 2-3 hours on ice. Incubation was terminated by rapid filtration on a multi-manifold tissue harvester (Brandel, Gaithersburg, Md.) using GF/B filters presoaked in 0.33% polyethyleneimine (w/v) to reduce non-specific binding. Tissue was rinsed 3 times in PBS, pH 7.4. Scintillation fluid was added to filters containing the washed tissue and allowed to equilibrate. Filters were then counted to determine radioactivity bound to the membranes by liquid scintillation counting (2200CA Tri-Carb LSC, Packard Instruments, 50% efficiency or Wallac Trilux 1450 MicroBeta, 40% efficiency, Perkin Elmer).

Data were expressed as disintegrations per minute (DPMs). Within each assay, each point had 2-3 replicates. The replicates for each point were averaged and plotted against the log of the drug concentration. IC₅₀, which is the concentration of the compound that produces 50% inhibition of binding, was determined by least squares non-linear regression. Ki values were calculated using the Cheng-Prussof equation (1973):

Ki=IC₅₀/(1+N/Kd)

where N is the concentration of [³H]nicotine and Kd is the affinity of nicotine (3 nM, determined in a separate experiment).

α7 nAChR Subtype

Preparation of membranes from rat hippocampus: Rats (female, Sprague-Dawley), weighing 150-250 g, were maintained on a 12 h light/dark cycle and were allowed free access to water and food supplied by PMI Nutrition International, Inc. Animals were anesthetized with 70% CO₂, then decapitated. Brains were removed and placed on an ice-cold platform. The hippocampus was removed and placed in 10 volumes (weight:volume) of ice-cold preparative buffer (137 mM NaCl, 10.7 mM KCl, 5.8 mM KH₂PO₄, 8 mM Na₂HPO₄, 20 mM HEPES (free acid), 5 mM iodoacetamide, 1.6 mM EDTA, pH 7.4); PMSF, dissolved in methanol to a final concentration of 100 μM, was added and the tissue suspension was homogenized by Polytron. The homogenate was centrifuged at 18,000×g for 20 min at 4° C. and the resulting pellet was re-suspended in 10 volumes of ice-cold water. After 60 min incubation on ice, a new pellet was collected by centrifugation at 18,000×g for 20 min at 4° C. The final pellet was re-suspended in 10 volumes of buffer and stored at −20° C.

On the day of the assay, tissue was thawed, centrifuged at 18,000×g for 20 min, and then re-suspended in ice-cold PBS (Dulbecco's Phosphate Buffered Saline, 138 mM NaCl, 2.67 mM KCl, 1.47 mM KH₂PO_{4,} 8.1 mM Na₂HPO₄, 0.9 mM CaCl₂, 0.5 mM MgCl₂, Invitrogen/Gibco, pH 7.4) to a final concentration of approximately 2 mg protein/mL. Protein was determined by the method of Lowry et al., J. Biol. Chem. 193: 265 (1951), herein incorporated by reference, using bovine serum albumin as the standard.

Assay: The binding of [³H]MLA was measured using a modification of the methods of Davies et al., Neuropharmacol. 38: 679 (1999), herein incorporated by reference. [³H]MLA (Specific Activity=25-35 Ci/mmol) was obtained from Tocris. The binding of [³H]MLA was determined using a 2 h incubation at 21° C. Incubations were conducted in 48-well micro-titre plates and contained about 200 μg of protein per well in a final incubation volume of 300 μL. The incubation buffer was PBS and the final concentration of [³H]MLA was 5 nM. The binding reaction was terminated by filtration of the protein containing bound ligand onto glass fiber filters (GF/B, Brandel) using a Brandel Tissue Harvester at room temperature. Filters were soaked in de-ionized water containing 0.33% polyethyleneimine to reduce non-specific binding. Each filter was washed with PBS (3×1 mL) at room temperature. Non-specific binding was determined by inclusion of 50 μM non-radioactive MLA in selected wells.

The inhibition of [³H]MLA binding by test compounds was determined by including seven different concentrations of the test compound in selected wells. Each concentration was replicated in triplicate. IC₅₀ values were estimated as the concentration of compound that inhibited 50 percent of specific [³H]MLA binding. Inhibition constants (Ki values), reported in nM, were calculated from the IC₅₀ values using the method of Cheng et al., Biochem. Pharmacol. 22: 3099-3108 (1973), herein incorporated by reference.

### Example 9

### Tabular Receptor Binding Data

Receptor binding data for compounds of the present invention are shown in Table 1.

TABLE 1

| | Rat | Human | |
|---|---|---|---|
| | α4β2 | α4β2 | Rat |
| Example | Kᵢ | Kᵢ | α7 Kᵢ |
| 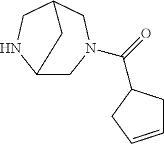 | 11 | 13 | 64 |
| 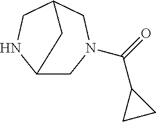 | 1.3 | 1.5 | 290 |
| 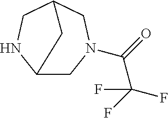 | 2400 | 2100 | >10,000 |
| 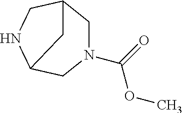 | 6.8 | 12 | ND |
| 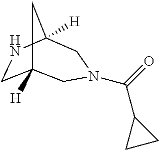 | 14 | 7.3 | 98 |
| 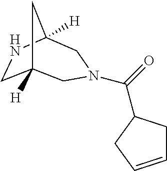 | 330 | 420 | 29 |
| 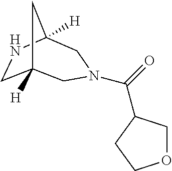 | 140 | 46 | ND |
| 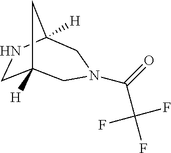 | ND | ND | ND |
| 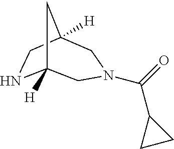 | 0.5 | 0.8 | 190 |
| 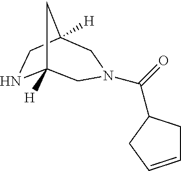 | 5.5 | 4.4 | 65 |
| 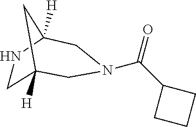 | 14 | 13 | 370 |
| 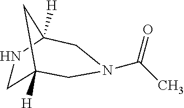 | 580 | 730 | ND |
| 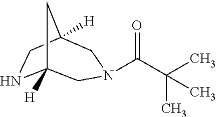 | 68 | 26 | ND |
| 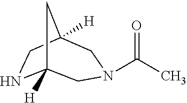 | 14 | 34 | ND |
| 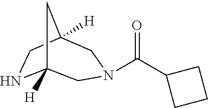 | 2.2 | 1.6 | 440 |
| 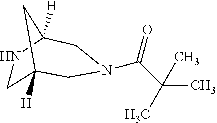 | ND | ND | ND |
| 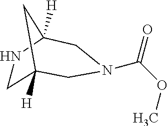 | 100 | 120 | ND |
| 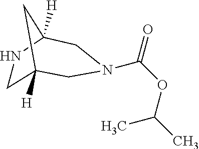 | 970 | 560 | ND |
| 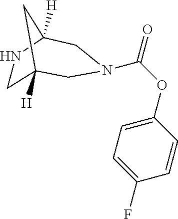 | ND | ND | ND |
| 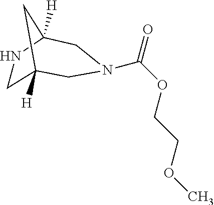 | 1000 | 580 | ND |
| 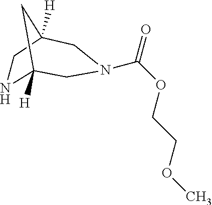 | 74 | 310 | ND |
| 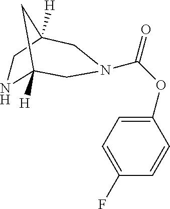 | 45 | 310 | ND |
| 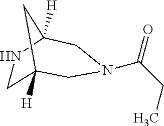 | 30 | 24 | 2000 |
| 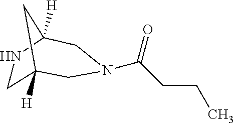 | 18 | 18 | 160 |
| 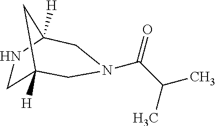 | 380 | 380 | 220 |
| 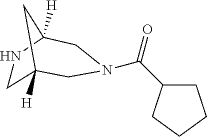 | 130 | 160 | 130 |
| 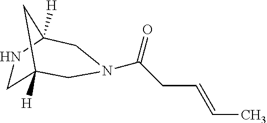 | 180 | 220 | ND |
| 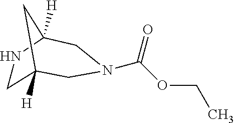 | 25 | 49 | ND |
| 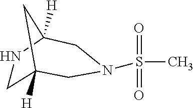 | ND | 2300 | ND |
| 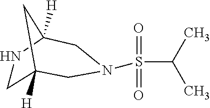 | 750 | 16 | ND |
| 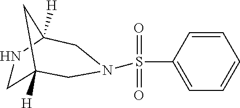 | ND | ND | ND |
| 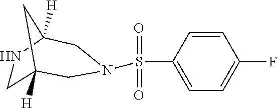 | ND | ND | ND |
| 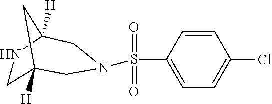 | ND | ND | ND |
| 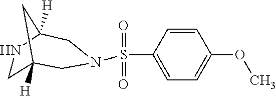 | 750 | 580 | ND |
| 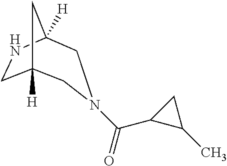 | 4.8 | 6.8 | 460 |
| 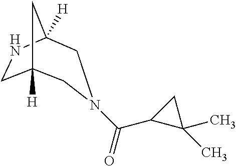 | 49 | 31 | ND |
| 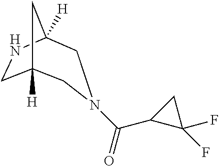 | 11 | 11 | 670 |
| 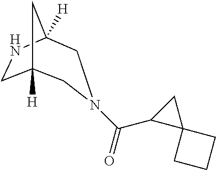 | 3.0 | 2.3 | ND |
| 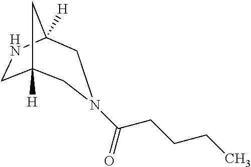 | 36 | 77 | ND |
| 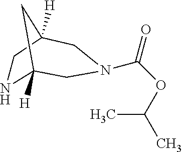 | 24 | 44 | ND |
| 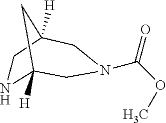 | 4.8 | 7.9 | ND |
| 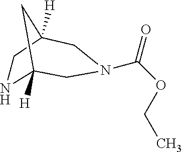 | 11 | 15 | 4200 |
| 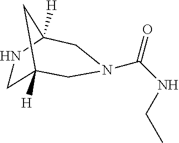 | 160 | 560 | ND |
| 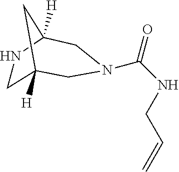 | 130 | 910 | ND |
| 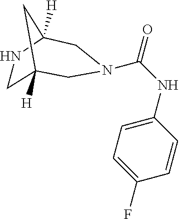 | 2200 | >10,000 | ND |
| 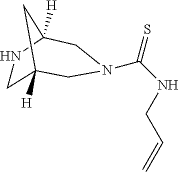 | 9900 | >10,000 | ND |
| 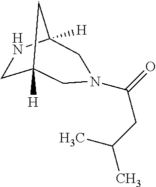 | 320 | 350 | ND |
| 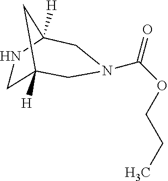 | 43 | 94 | ND |
| 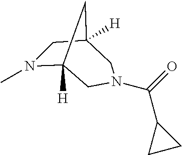 | 19 | 18 | ND |
| 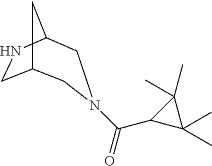 | ND | 8600 | ND |
| 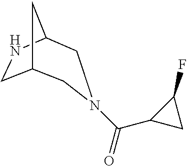 | 22 | 14 | 260 |
| 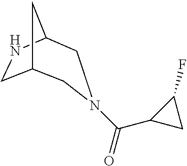 | 21 | 11 | 630 |
| 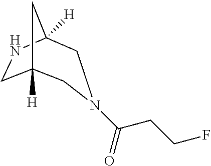 | 31 | 27 | 950 |
| 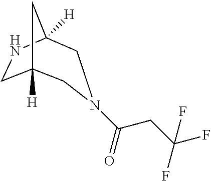 | 110 | 73 | ND |
| 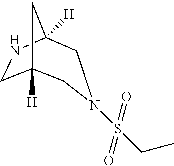 | ND | 1200 | ND |
| 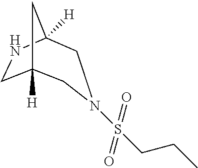 | ND | 2800 | ND |
| 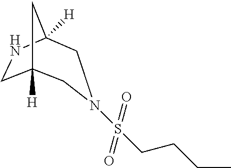 | ND | 2100 | ND |
| 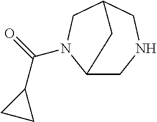 | 5.6 | 3.4 | ND |
| 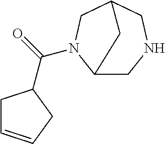 | 120 | 21 | 160 |
| 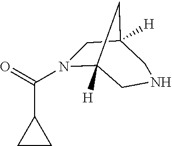 | 7.0 | 4.0 | ND |
| 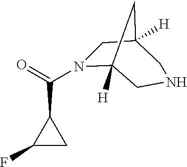 | 2.9 | 3.7 | ND |
| 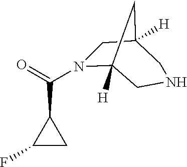 | ND | 11 | ND |
| 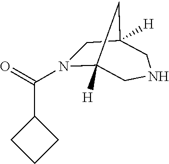 | ND | 23 | ND |
| 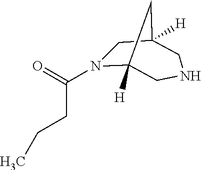 | ND | 34 | ND |
| 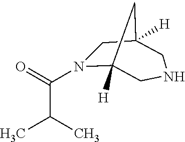 | ND | 590 | ND |
| 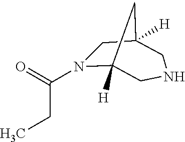 | ND | 27 | ND |
| 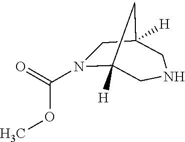 | 40 | 86 | ND |
| 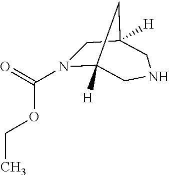 | 32 | 78 | >10,000 |
| 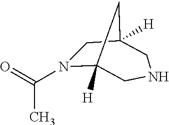 | ND | 1300 | ND |
| 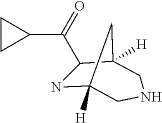 | ND | 480 | 1300 |
| 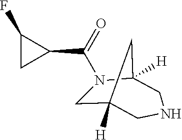 | 170 | 68 | 1300 |
| 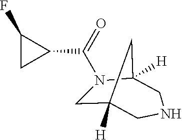 | ND | 230 | ND |
| 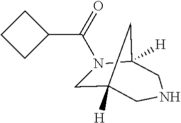 | ND | 180 | 1500 |
| 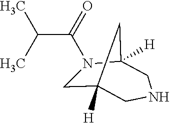 | ND | 770 | 890 |
| 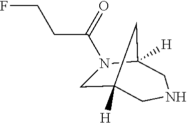 | ND | 2400 | 2300 |
| 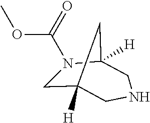 | ND | 5700 | ND |
| 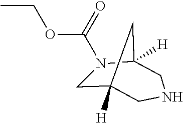 | ND | 1400 | ND |
| 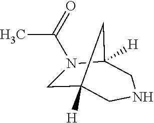 | ND | ND | ND |

### Summary of Biological Data

Compounds of Table 1, representative of the present invention, exhibited inhibition constants (Ki values) at the rat and human α4β2 subtypes in the ranges of 0.5 nM to 9,900 nM and 0.8 nM to >10,000 nM respectively, indicating affinity for the α4β2 subtype. Ki values at the α7 subtype vary within the range of 29 nM to >10,000 nM, indicating affinity for the α7 subtype. The notation “ND” means that the Ki value was not determined. In some cases, this was a result of the assay being unavailable for a period of time, and in other cases, this was because the compounds failed to bind sufficiently in high through-put screening (HTS) to warrant Ki determination. This latter situation was much more common for binding at the α7 subtype, as compared to the α4β2 subtype.

In this regard, failing to bind sufficiently in HTS means, for the α4β2 subtype, that the compound failed to inhibit, at 5 μM concentration, the binding of 5 nM ³H-nicotine by at least 50%, and for the α7 subtype, that the compound failed to inhibit, at 5 μM concentration, the binding of 5 nM ³H-MLA (methyllycaconitine) by at least 50%.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A compound as represented by either Formula I or Formula II: 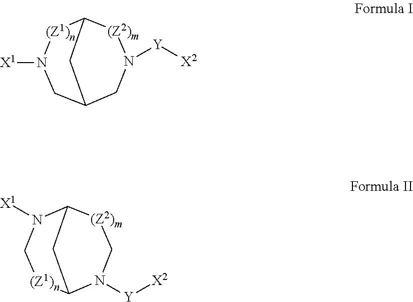
wherein:
Y is C(O), C(S), or S(O)_{q};
q is 1 or 2;
Z¹ is methylene and n is 0 or 1;
Z² is methylene and m is 0 or 1;
when n is 0, then m is 1;
when m is 0, then n is 1;
X¹ is hydrogen or C₁₋₆ alkyl;
X² is R^{I}, OR^{II}, or NR^{III}R^{IV};
when Y is C(O), then R^{I} is hydrogen, optionally substituted C₁₋₆ alkyl, optionally substituted C₃₋₈ cycloalkyl, optionally substituted C₂₋₆alkenyl, optionally substituted C₃₋₈cycloalkenyl, optionally substituted C₂₋₆ alkynyl, optionally substituted heterocyclyl, optionally substituted aryl, optionally substituted arylalkyl, or optionally substituted heteroarylalkyl;
when Y is C(S) or S(O)_{q}, then R^{I} is hydrogen, optionally substituted C₁₋₆ alkyl, optionally substituted C₃₋₈ cycloalkyl, optionally substituted C₂₋₆ alkenyl, optionally substituted C₃₋₈ cycloalkenyl, optionally substituted C₂₋₆ alkynyl, optionally substituted heterocyclyl, optionally substituted aryl, optionally substituted heteroaryl, optionally substituted arylalkyl, or optionally substituted heteroarylalkyl;
R^{II} is hydrogen, optionally substituted C₁₋₆ alkyl, optionally substituted C₃₋₈ cycloalkyl, optionally substituted C₂₋₆ alkenyl, optionally substituted C₃₋₈ cycloalkenyl, optionally substituted C₂₋₆ alkynyl, optionally substituted heterocyclyl, optionally substituted aryl, optionally substituted heteroaryl, optionally substituted arylalkyl, or optionally substituted heteroarylalkyl; and
each of R^{III} and R^{IV} are individually hydrogen, optionally substituted C₁₋₆ alkyl, optionally substituted C₃₋₈ cycloalkyl, optionally substituted C₂₋₆ alkenyl, optionally substituted C₃₋₈ cycloalkenyl, optionally substituted C₂₋₆alkynyl, optionally substituted heterocyclyl, optionally substituted aryl, optionally substituted heteroaryl, optionally substituted arylalkyl, or optionally substituted heteroarylalkyl;
or R^{III} and R^{IV} can combine with the nitrogen to which they are attached to form a 3- to 8-membered ring that may contain one or more degrees of unsaturation and may contain one or more additional heteroatom selected from N, O, or S;
where the term “optionally substituted” refers to optional substitution of one or more hydrogen atoms by a substituent independently selected from C₁₋₆ alkyl, C₃₋₈ cycloalkyl, heterocyclyl, aryl, heteroaryl, halogen, OR^{V}, NR^{V}R^{VI}, C₁₋₆ haloalkyl, —CN, —NO₂, —C₂R^{V}, —SR^{V}, —N₃, —C(═O)NR^{V}R^{VI}, —NR^{V}C(═O)R^{VI}, —OC(═O)NR^{V}R^{VI}, —NR^{V}C(═O)OR^{VI}, —SO₂R^{V}, —SO₂NR^{V}R^{VI}, and —NR^{V}SO₂R^{VI}, where R^{V} and R^{VI} are individually hydrogen, C₁₋₆ alkyl, C₃₋₈ cycloalkyl, heterocyclyl, aryl, or arylalkyl;
or a pharmaceutically acceptable salt thereof.

2. A compound selected from the group consisting of:
3-(cyclopentene-4-ylcarbonyl)-3,6-diazabicyclo[3.2.1]octane,
3-(cyclopropylcarbonyl)-3,6-diazabicyclo[3.2.1]octane,
3-(trifluoroacetyl)-3,6-diazabicyclo[3.2.1]octane,
3-(methoxycarbonyl)-3,6-diazabicyclo[3.2.1]octane,
(1S,5S)-3-(cyclopropylcarbonyl)-3,6-diazabicyclo[3.2.1]octane,
(1S,5S)-3-(cyclopentene-4-ylcarbonyl)-3,6-diazabicyclo[3.2.1]octane,
(1S,5S)-3-(tetrahydrofuran-3-ylcarbonyl)-3,6-diazabicyclo[3.2.1]octane,
(1S,5S)-3-(trifluoroacetyl)-3,6-diazabicyclo[3.2.1]octane,
(1R,5R)-3-(cyclopropylcarbonyl)-3,6-diazabicyclo[3.2.1]octane,
(1R,5R)-3-(cyclopentene-4-ylcarbonyl)-3,6-diazabicyclo[3.2.1]octane,
(1S,5S)-3-(cyclobutylcarbonyl)-3,6-diazabicyclo[3.2.1]octane,
(1S,5S)-3-(acetyl)-3,6-diazabicyclo[3.2.1]octane,
(1R,5R)-3-(trifluoroacetyl)-3,6-diazabicyclo[3.2.1]octane,
(1R,5R)-3-(acetyl)-3,6-diazabicyclo[3.2.1]octane,
(1R,5R)-3-(cyclobutylcarbonyl)-3,6-diazabicyclo[3.2.1]octane,
(1S,5S)-3-(2,2-dimethylpropanoyl)-3,6-diazabicyclo[3.2.1]octane,
(1R,5R)-3-(2,2-dimethylpropanoyl)-3,6-diazabicyclo[3.2.1]octane,
(1S,5S)-3-(methoxycarbonyl)-3,6-diazabicyclo[3.2.1]octane,
(1S,5S)-3-(isopropoxycarbonyl)-3,6-diazabicyclo[3.2.1]octane,
(1S,5S)-3-(4-fluorophenoxycarbonyl)-3,6-diazabicyclo[3.2.1]octane,
(1S,5S)-3-(2-methoxyethoxycarbonyl)-3,6-diazabicyclo[3.2.1]octane,
(1R,5R)-3-(2-methoxyethoxycarbonyl)-3,6-diazabicyclo[3.2.1]octane,
(1R,5R)-3-(4-fluorophenoxycarbonyl)-3,6-diazabicyclo[3.2.1]octane,
(1S,5S)-3-(propanoyl)-3,6-diazabicyclo[3.2.1]octane,
(1S,5S)-3-(butanoyl)-3,6-diazabicyclo[3.2.1]octane,
(1S,5S)-3-(2-methylpropanoyl)-3,6-diazabicyclo[3.2.1]octane,
(1S,5S)-3-(cyclopentylcarbonyl)-3,6-diazabicyclo[3.2.1]octane,
E-(1S,5S)-3-(3-pentenoyl)-3,6-diazabicyclo[3.2.1]octane,
(1S,5S)-3-(ethoxycarbonyl)-3,6-diazabicyclo[3.2.1]octane,
(1S,5S)-3-(methylsulfonyl)-3,6-diazabicyclo[3.2.1]octane,
(1S,5S)-3-(isopropylsulfonyl)-3,6-diazabicyclo[3.2.1]octane,
(1S,5S)-3-(phenylsulfonyl)-3,6-diazabicyclo[3.2.1]octane,
(1S,5S)-3-(4-fluorophenylsulfonyl)-3,6-diazabicyclo[3.2.1]octane,
(1S,5S)-3-(4-chlorophenylsulfonyl)-3,6-diazabicyclo[3.2.1]octane,
(1S,5S)-3-(4-methoxyphenylsulfonyl)-3,6-diazabicyclo[3.2.1]octane,
(1S,5S)-3-(2-methylcyclopropylcarbonyl)-3,6-diazabicyclo[3.2.1]octane,
(1S,5S)-3-(2,2-dimethylcyclopropylcarbonyl)-3,6-diazabicyclo[3.2.1]octane,
(1S,5S)-3-(2,2-difluorocyclopropylcarbonyl)-3,6-diazabicyclo[3.2.1]octane,
(1S,5S)-3-(spiro[2.3]hexan-1-ylcarbonyl)-3,6-diazabicyclo[3.2.1]octane,
(1S,5S)-3-(pentanoyl)-3,6-diazabicyclo[3.2.1]octane,
(1R,5R)-3-(isopropoxycarbonyl)-3,6-diazabicyclo[3.2.1]octane,
(1R,5R)-3-(methoxycarbonyl)-3,6-diazabicyclo[3.2.1]octane,
(1R,5R)-3-(ethoxycarbonyl)-3,6-diazabicyclo[3.2.1]octane,
(1S,5S)-3-(N-ethylcarbamoyl)-3,6-diazabicyclo[3.2.1]octane,
(1S,5S)-3-(N-allylcarbamoyl)-3,6-diazabicyclo[3.2.1]octane,
(1S,5S)-3-(N-(4-fluorphenyl)carbamoyl)-3,6-diazabicyclo[3.2.1]octane,
(1S,5S)-3-(N-allylthiocarbamoyl)-3,6-diazabicyclo[3.2.1]octane,
(1S,5S)-3-(3-methylbutanoyl)-3,6-diazabicyclo[3.2.1]octane,
(1S,5S)-3-(n-propoxycarbonyl)-3,6-diazabicyclo[3.2.1]octane,
(1R,5R)-3-(cyclopropylcarbonyl)-6-methyl-3,6-diazabicyclo[3.2.1]octane,
(1S,5S)-3-(2,2,3,3-tetramethylcyclopropylcarbonyl)-3,6-diazabicyclo[3.2.1]octane,
(1S,5S)-3-(cis-2-fluorocyclopropylcarbonyl)-3,6-diazabicyclo[3.2.1]octane,
(1S,5S)-3-(trans-2-fluorocyclopropylcarbonyl)-3,6-diazabicyclo[3.2.1]octane,
(1S,5S)-3-(3-fluoropropanoyl)-3,6-diazabicyclo[3.2.1]octane,
(1S,5S)-3-(3,3,3-trifluoropropanoyl)-3,6-diazabicyclo[3.2.1]octane,
(1S,5S)-3-(ethylsulfonyl)-3,6-diazabicyclo[3.2.1]octane,
(1S,5S)-3-(n-propylsulfonyl)-3,6-diazabicyclo[3.2.1]octane,
(1S,5S)-3-(n-butylsulfonyl)-3,6-diazabicyclo[3.2.1]octane,
6-(cyclopropylcarbonyl)-3,6-diazabicyclo[3.2.1]octane,
6-(cyclopentene-4-ylcarbonyl)-3,6-diazabicyclo[3.2.1]octane,
(1S,5R)-6-(cyclopropylcarbonyl)-3,6-diazabicyclo[3.2.1]octane,
(1S,5R)-6-(cis-2-fluorocyclopropylcarbonyl)-3,6-diazabicyclo[3.2.1]octane,
(1S,5R)-6-(trans-2-fluorocyclopropylcarbonyl)-3,6-diazabicyclo[3.2.1]octane,
(1S,5R)-6-(cyclobutylcarbonyl)-3,6-diazabicyclo[3.2.1]octane,
(1S,5R)-6-(butanoyl)-3,6-diazabicyclo[3.2.1]octane,
(1S,5R)-6-(2-methylproanoyl)-3,6-diazabicyclo[3.2.1]octane,
(1S,5R)-6-(propanoyl)-3,6-diazabicyclo[3.2.1]octane,
(1S,5R)-6-(methoxycarbonyl)-3,6-diazabicyclo[3.2.1]octane,
(1S,5R)-6-(ethoxycarbonyl)-3,6-diazabicyclo[3.2.1]octane,
(1S,5R)-6-(acetyl)-3,6-diazabicyclo[3.2.1]octane,
(1R,5S)-6-(cyclopropylcarbonyl)-3,6-diazabicyclo[3.2.1]octane,
(1R,5S)-6-(cis-2-fluorocyclopropylcarbonyl)-3,6-diazabicyclo[3.2.1]octane,
(1R,5S)-6-(trans-2-fluorocyclopropylcarbonyl)-3,6-diazabicyclo[3.2.1]octane,
(1R,5S)-6-(cyclobutylcarbonyl)-3,6-diazabicyclo[3.2.1]octane,
(1R,5S)-6-(2-methylpropanoyl)-3,6-diazabicyclo[3.2.1]octane,
(1R,5S)-6-(3-fluoropropanoyl)-3,6-diazabicyclo[3.2.1]octane,
(1R,5S)-6-(methoxycarbonyl)-3,6-diazabicyclo[3.2.1]octane,
(1R,5S)-6-(ethoxycarbonyl)-3,6-diazabicyclo[3.2.1]octane, and
(1R,5S)-6-(acetyl)-3,6-diazabicyclo[3.2.1]octane,
or a pharmaceutically acceptable salt thereof

3. A method for treatment of central nervous system disorders and dysfunctions, comprising administering to a mammal in need of such treatment, a therapeutically effective amount of the compound according to claim 1.

4. A method for treatment of central nervous system disorders and dysfunctions, comprising administering to a mammal in need of such treatment, a therapeutically effective amount of the compound according to claim 2.

5. The method of claim 4, wherein the disorder is selected from the group consisting of age-associated memory impairment, mild cognitive impairment, pre-senile dementia, early onset Alzheimer's disease, senile dementia, dementia of the Alzheimer's type, Lewy body dementia, vascular dementia, Alzheimer's disease, stroke, AIDS dementia complex, attention deficit disorder, attention deficit hyperactivity disorder, dyslexia, schizophrenia, schizophreniform disorder, schizoaffective disorder, cognitive deficits in schizophrenia, and cognitive dysfunction in schizophrenia.

6. The method of claim 5, wherein the disorder is selected from the group consisting of mild to moderate dementia of the Alzheimer's type, attention deficit disorder, attention deficit hyperactivity disorder, mild cognitive impairment, age-associated memory impairment, cognitive deficits in schizophrenia, and cognitive dysfunction in schizophrenia.

7. A pharmaceutical composition comprising a compound according to claim 1, and one or more pharmaceutically acceptable carrier.

8. A pharmaceutical composition comprising a compound according to claim 2, and one or more pharmaceutically acceptable carrier.

9. A method of treating inflammation, the inflammatory response associated with bacterial and/or viral infection, pain, metabolic syndrome, autoimmune disorders, addictions, and obesity, comprising administering to a mammal in need of such treatment, a therapeutically effective amount of the compound according to claim 1.

10. A method of treating inflammation, the inflammatory response associated with bacterial and/or viral infection, pain, metabolic syndrome, autoimmune disorders, addictions, and obesity, comprising administering to a mammal in need of such treatment, a therapeutically effective amount of the compound according to claim 2.

11. The method of claim 3, wherein the disorder is selected from the group consisting of age-associated memory impairment, mild cognitive impairment, pre-senile dementia, early onset Alzheimer's disease, senile dementia, dementia of the Alzheimer's type, Lewy body dementia, vascular dementia, Alzheimer's disease, stroke, AIDS dementia complex, attention deficit disorder, attention deficit hyperactivity disorder, dyslexia, schizophrenia, schizophreniform disorder, schizoaffective disorder, cognitive deficits in schizophrenia, and cognitive dysfunction in schizophrenia.

12. The method of claim 11, wherein the disorder is selected from the group consisting of mild to moderate dementia of the Alzheimer's type, attention deficit disorder, attention deficit hyperactivity disorder, mild cognitive impairment, age-associated memory impairment, cognitive deficits in schizophrenia, and cognitive dysfunction in schizophrenia.
